SEQUENCE LISTING (28 sequences)

SEQ ID NO: 1 - protein, 103 aa, unidentified
VRIFAGNDPAHTATGSSGISSPTPALTPLMLDEATGKLVVWDGQKAGSAVGILVLPLEGTETALTYYKSGTFATEAIHWPESVDEHKKANAFAGSALSHAALP

SEQ ID NO: 2 - protein, 157 aa, synthetic construct
RSDLGKKLLEAARAGQDDEVRILMANGADANAYDHYGRTPLHMAAAVGHLEIVEVLLRNGADVNAVDTNGTTPLHLAASLGHLEIVEVLLKYGADVNAKDATGITPLYLAAYWGHLEIVEVLLKHGADVNAQDKFGKTPFDLAIDNGNEDIAEVLQG

SEQ ID NO: 3 - protein, 760 aa, Homo sapiens
MKTWVKIVFGVATSAVLALLVMCIVLRPSRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPVVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEEYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSD

SEQ ID NO: 4 - protein, 761 aa, Mus musculus
MKTWLKTVFGVTTLAALALVVICIVLRPSRVYKPEGNTKRALTLKDILNGTFSYKTYFPNWISEQEYLHQSEDDNIVFYNIETRESYIILSNSTMKSVNATDYGLSPDRQFVYLESDYSKLWRYSYTATYYIYDLQNGEFVRGYELPRPIQYLCWSPVGSKLAYVYQNNIYLKQRPGDPPFQITYTGRENRIFNGIPDWVYEEEMLATKYALWWSPDGKFLAYVEFNDSDIPIIAYSYYGDGQYPRTINIPYPKAGAKNPVVRVFIVDTTYPHHVGPMEVPVPEMIASSDYYFSWLTWVSSERVCLQWLKRVQNVSVLSICDFREDWHAWECPKNQEHVEESRTGWAGGFFVSTPAFSQDATSYYKIFSDKDGYKHIHYIKDTVENAIQITSGKWEAIYIFRVTQDSLFYSSNEFEGYPGRRNIYRISIGNSPPSKKCVTCHLRKERCQYYTASFSYKAKYYALVCYGPGLPISTLHDGRTDQEIQVLEENKELENSLRNIQLPKVEIKKLKDGGLTFWYKMILPPQFDRSKKYPLLIQVYGGPCSQSVKSVFAVNWITYLASKEGIVIALVDGRGTAFQGDKFLHAVYRKLGVYEVEDQLTAVRKFIEMGFIDEERIAIWGWSYGGYVSSLALASGTGLFKCGIAVAPVSSWEYYASIYSERFMGLPTKDDNLEHYKNSTVMARAEYFRNVDYLLIHGTADDNVHFQNSAQIAKALVNAQVDFQAMWYSDQNHGISSGRSQNHLYTHMTHFLKQCFSLSD

SEQ ID NO: 5 - protein, 121 aa, synthetic construct
DLGKKLLDAARAGQDDEVRILMANGADVNAMDVWGVTPLHLAAWHRHLEIVEVLLKTGADVNANDDSGFTPLHLAAHWGHLEIVEVLLKHGADVNAQDKFGKTPFDLAIDNGNEDIAEVLQ

SEQ ID NO: 6 - protein, 154 aa, synthetic construct
DLGKKLLEAARAGQDDEVRILMANGADVNAQDMTGKTPLHLAAREGHLEIVEVLLKTGADVNAKDLWGVTPLHLAAFSGHLEIVEVLLKTGADVNAWDWEGKTPLHLAAVKGHPEIVEVLLKHGADVNAQDKFGKTPFDLAIDNGNEDIAEVLQ

SEQ ID NO: 7 - protein, 154 aa, synthetic construct
DLGKKLLDAASVGQDDEVRILMANGADVNASDTYGRTPLHVAAQHGHLEIVDVLLAHGADVNASNAYGSTPLHVAAWTGHLEIVDVLLAYGADVNASNSQGNTPLHAAAATGHLEIVEVLLKHGADVNAQDKFGKTPFDLAIDNGNEDIAEVLQ

SEQ ID NO: 8 - protein, 154 aa, synthetic construct
DLGKKLLEAARAGQDDEVRILMANGADVNAEDHVGRTPLHLAAAHGHLEIVEVLLKTGADVNARDRHGITPLHLAAWSGHLEIVEVLLKTGADVNASDKQGNTPLHLAAYRGHLEIVEVLLEAGADVNAQDKFGKTPFDLAIDNGNEDIAEVLQ

SEQ ID NO: 9 - protein, 154 aa, synthetic construct
DLGKKLLEAARAGQDDEVRILMANGADVNASDNWGRTPLHVAAQRGHLEIVDVLLAHGADDNASNRWGSTPLHVAAYTGHLEIVDVLLANGADVNASTANGETPLHAAAHAGHLEIVDVLLKHGADVNAQDKFGKTPFDLAIDNGNEDIAEVLQ

SEQ ID NO: 10 - protein, 121 aa, synthetic construct
DLGKKLLXAARAGQDDEVRILMANGADVNAXDXFGXTPLHLAAWXGHLEIVEVLLKAGADVNAXDXWGNTPLHLAAHRGHLEIVEVLLKHGADVNAQDKFGKTPFDLAIDNGNEDIAEVLQ

SEQ ID NO: 11 - protein, 121 aa, synthetic construct
DLGKKLLEAARAGQDDEVRILMANGADVNAADEYGITPLHLAAFFGHLEIVEVLLKTGADVNADDAWGSTPLHLAAYKGHLEIVEVLLKHGADVNAQDKFGKTPFDLAIDNGNEDIAEVLQ

SEQ ID NO: 12 - protein, 121 aa, synthetic construct
DLGKKLLEAARAGQDDEVRILMANGADVNAXDWWGITPLHLAAXVGHLEIVEVLLKTGADVNABDKXGXTPLHLAAYXGHLEIVEVLLXXGADVNAZDKFGKTPFDLAIDNGNEDIAEVLQ

SEQ ID NO: 13 - protein, 154 aa, synthetic construct
DLGKKLLEAARAGQDDEVRILMANGADVNAADDQGRTPLHLAAMHGHLEIVEVLLKTGADVNANDWFGITPLHLAAWWGHLEIVEVLLKAGADVNAVDHSGDTPLHLAAHLGHLEIVEVLLKHGADVNAQDKFGKTPFDLAIDNGNEDIAEVLQ

SEQ ID NO: 14 - protein, 121 aa, synthetic construct
DLVKKLLEAARAGQDDEVRILMANGADVNANDHFGITPLHLTAWWGHLEIVEVLLKAGADVNAVDHWGTTPLHLAAHQGHLEIVEVLLKHGADVNAQDKFGKTAFDISIDNGNEDIAEVLQ

SEQ ID NO: 15 - protein, 121 aa, synthetic construct
DLGKKLLEAARAGQDDEVRILMANGADVNAQDQRGRTPLHLAAAWGHLEIVEVLLKTGADVNAADDMGHTPLHLAAAFGHLEIVEVLLKHGADVNAQDKFGKTPFDLAIDNGNEDIAEVLQ

SEQ ID NO: 16 - protein, 154 aa, synthetic construct
DLGKKLLEAARAGQDDEVRILMANGADVNATDQHGRTPLHLAAHTGHLEIVEVLLKTGADVNAQDHQGITPLHLAAFKGHLEIVEVLLKTGADVNAQDNWGYTPLHLAAARGHLEIVEVLLKAGADVNAQDKFGKTPFDLAIDNGNEDIAEVLQ

SEQ ID NO: 17 - protein, 154 aa, synthetic construct
DLGKKLLEAARAGQDDEVRILMANGADVNAMDMWGITPLHLAAWAGHLEIVEVLLKTGADVNAFDQHGNTPLHLAAYRGHLEIVEVLLKTGADVNAHDNWGTTPLHLTAHNGHLEIVEVLLKAGADVNAQDKFGKTPFDLAIDNGNEDIAEVLQ

SEQ ID NO: 18 - protein, 121 aa, synthetic construct
DLGKKLLEAARAGQDDEVRILMANGADVNAKDRYGITPLHLAAYWGHLEIVEVLLKAGSDVNALDYHGSTPLHLAAWFGHLEIVEVLLKHGADVNAQDKFGKTPFDLAIDNGNEDIAEVLQ

SEQ ID NO: 19 - protein, 154 aa, synthetic construct
DLGKKLLEAARAGQDDEVRILMANGADVNAADNYGITPLHLAAYRGHLEIVEVLLKTGADVNAHDNVGNTPLHLAAAKGHLEIVEVLLKAGADVNADDVQGVTPLHLAASQGHLEIVEVLLKHGADVNAQDKFGKTPFDLAIDNGNEDIAEVLQ

SEQ ID NO: 20 - protein, 121 aa, synthetic construct
DLGKKLLEAARAGQDDEVRILMANGADVNAVDHYGITPLHLAAFWGHLEIVEVLLKTGADVNAXDXWGWTPLHLAAVRGHLEIVEVLLKHGADVNAQDKFGKTPFDLAIDNGNEDIAEVLQ

SEQ ID NO: 21 - protein, 121 aa, synthetic construct
DLGKKLLEAARAGQDDEVRILMANGADVNAXDWXGITPLHLAAFXGHLEIVEVLLKAGADVNAVDSDGSTPLHLAAYXGHLEIVEVLLKHGADVNAQDKFGKTPFDLAIDNGNEDIAEVLQ

SEQ ID NO: 22 - protein, 154 aa, synthetic construct
DLGKKLLEAARAGQDDEVRILMANGADVNAVDYQGRTPLHLAAQYGHLEIVEVLLKTGADVNAQDHHGITPLHLAAFWGHLEIVEVLLKAGADVNAKDDQGNTPLHLAAWRGHLEIVEVLLKHGADVNAQDKFGKTPFDLAIDNGNEDIAEVLQ

SEQ ID NO: 23 - protein, 154 aa, synthetic construct
DLGKKLLEAAELGQLDEVRILMANGADVNAMDQHGITPLHLAAWHGHLEIVEVLLKTGADVNAKDQDGSTPLHLAAYLGHLEIVEVLLKTGADVNAFDKFGNTPLHLAAHRGHLEIVEVLLKHGADVNAQDKFGKTPFDLAIDNGNEDIAEVLQ

SEQ ID NO: 24 - protein, 121 aa, synthetic construct
DLGKKLLEAARAGQDDEVRILMANGADVNASDWWGITPLHLAAYWGHLEIVEVLLKTGADVNAQDNQGITPLHLAATLGHLEIVEVLLKHGADVNAQDKFGKTPFDLAIDNGNEDIAEVLQ

SEQ ID NO: 25 - protein, 121 aa, synthetic construct
DLGKKLLEAARYGQDDEVRILMANGADVNAEDQHGHTPLHLAAFSGHLEIVEVLLKTGADVNANDINGVTPLHLAANVGHLEIVEVLLKHGADVNAQDKFGKTPFDLAAYLGNEDIAEVLQ

SEQ ID NO: 26 - protein, 121 aa, synthetic construct
DLGKKLLEAARAGQDDEVRILMANGADVNAIDVHGITPLHLAAYWGHLEIVEVLLKAGADVNAEDESGHTPLHLAAHWGHLEIVEVLLKHGADVNAQDKFGKTPFDLAIDNGNEDIAEVLQ

SEQ ID NO: 27 - protein, 154 aa, synthetic construct
DLGKKLLEAARAGQDDEVRILMANGADVNATDWQGSTPLHLAAQHGHLEIVEVLLKTGADVNAEDQNGITPLHLAAYWGHLEIVEVLLKAGADVNAVDRQGNTPLHLAAVWGHLEIVEVLLKHGADVNAQDKFGKTPFDLAIDNGNEDIAEVLQ

SEQ ID NO: 28 - protein, 121 aa, synthetic construct
DLGKKLLEAARAGQDDEVRILMANGADVNAIDWWGITPLHLAALFGHLEIVEVLLKTGADVNAEDRLGFTPLHLAATTGHLEIVEVLLKAGADVNAQDKFGKTPFDLAIDNGNEDIAEVLQ

(19) United States
(12) Patent Application Publication
(10) Pub. No.: US 2026/0103541 A1
(43) Pub. Date: Apr. 16, 2026

(54) Paracrine Adenoviral Delivery of Biomolecules

(71) Applicant: Universität Zürich, Zürich (CH)

(72) Inventors: Karen Patricia HARTMANN, Zürich (CH); Andreas PLÜCKTHUN, Zürich (CH)

(21) Appl. No.: 19/117,140

(22) PCT Filed: Oct. 6, 2023
(86) PCT No.: PCT/EP2023/077775

§ 371 Date: Mar. 31, 2025

(30) Foreign Application Priority Data
Oct. 7, 2022 (EP) 22200275.0

(51) Int. Cl. C07K 16/40; A61K 39/00; A61K 48/00; C07K 16/32; C12N 15/86

(52) U.S. Cl. CPC C07K 16/40; A61K 48/0025; C07K 16/32; C12N 15/86; C12Y 304/21026; A61K 2039/505; C07K 2317/33; C07K 2318/00; C12N 2710/10043

(57) ABSTRACT

Disclosed herein are designed ankyrin repeat domain specifically binding to fibroblast activation protein (FAP). Such designed ankyrin repeat domains can be used in recombinant adapter molecules that are displayed on adenoviruses. Adenoviruses armed with such adapters efficiently transduce human fibroblasts and significantly broaden treatment opportunities through paracrine delivery of cargo to the tumor microenvironment (TME).

### FIELD OF THE INVENTION

Disclosed herein are designed ankyrin repeat protein domains specifically binding to fibroblast activation protein (FAP). Such designed ankyrin repeat protein domains can be used in recombinant adapter molecules that are displayed on adenoviruses. Adenoviruses armed with such adapters efficiently transduce human fibroblasts and significantly broaden treatment opportunities through paracrine delivery of cargo to the tumor microenvironment (TME).

### BACKGROUND

Immunotherapeutic approaches involving monoclonal antibodies and recombinant cytokines with anti-tumor activity have shown great success in many types of cancer. However, clinical application of these therapeutics, particularly of cytokine-based drugs, often remains restricted due to severe dose-limiting toxicities, as well as off-target effects, both hampering therapeutic efficacy and safety (J Interferon Cytokine Res (2019) 39:6-21). One promising strategy to overcome these obstacles and exploit the full potential of cancer immunotherapy is to utilize suitable delivery systems, such as viral vectors. The most commonly used viral vector is derived from human adenovirus serotype 5 (HAdV-C5) (J Gene Med (2018) 20: e3015). HAdV-C5-based vectors have shown to be superior to other clinically used viral vectors, mainly derived from lentivirus or adeno-associated virus, because of their following characteristics: (i) they do not integrate into the host cell genome and are therefore safe, (ii) they efficiently transduce dividing and non-dividing cells, and (iii) they have a large packaging capacity of up to 36 kilobase pairs (kb) (Curr Gene Ther (2002) 2:111-33; Gene Ther (2012) 19:109-13). The large packaging capacity allows for a simultaneous delivery of multiple payload genes, rendering adenoviral vectors very attractive for combination cancer immunotherapy.

Although HAdV-C5-based vectors harbor many favorable features that qualify them for applications in humans, they have shown compromised delivery efficacy because vector targeting to the tissue of interest remains challenging, especially upon intravenous administration. One hurdle is the strong liver tropism of HAdV-C5, shown to result from interactions of viral capsid proteins with, for example, blood components like blood coagulation factor X (FX) (Gene Ther (2012) 19:109-13; Proc Natl Acad Sci U S A (2008) 105:5483-8; Mol Ther (2008) 16:1474-80). Another hurdle is the effective ablation of the virion's natural cell specificity and the redirection of the vector to the desired tissue. This requires disruption of interactions with cell surface receptors that naturally mediate viral cell entry, while introducing specificity for the cell type of choice. Here the major adenovirus capsid proteins hexon, penton base, and fiber play a crucial role (see FIG. 1) (J Gen Virol (2009) 90 (Pt 1): 1-20). For cell entry, the fiber knob of HAdV-C5 binds to its primary attachment receptor Coxsackievirus and Adenovirus Receptor (CAR), then the penton base interacts with integrin receptors, which initiates cellular virus uptake by clathrin-mediated endocytosis (Rev Med Virol (2009) 19:165-78; Trends Pharmacol Sci (2012) 33:442-8; Cell Microbiol (2013) 15:16-23; J Gene Med (2003) 5:451-62).

Numerous approaches aiming at modifying undesired virion interactions have been tested. These have been mainly based on manipulations of the viral capsid through, for example, genetic mutations, as for instance deletion of a binding motif on the hexon hypervariable region 7 (HVR7) known to account for FX-mediated liver tropism (Blood (2009) 114:965-71). Furthermore, chemical capsid modifications, and genetic fusions of targeting peptides mainly to the fiber have been used for redirection to target cells (Curr Gene Ther (2011) 11:241-58; Hum Gene Ther (2006) 17:264-79; Mol Ther (2000) 1:391-405; Mol Ther (2005) 12:384-93; Mol Pharm (2005) 2:341-7). However, most attempts led to reduced transduction efficiencies and did not achieve sufficient liver detargeting.

To improve vector specificity, the inventors developed a generic retargeting system which is based on a bispecific adapter module (Proc Natl Acad Sci USA (2013) 110: E869-77; J Mol Biol (2011) 405:410-26). The adapter is composed of a central Designed Ankyrin Repeat Protein (DARPin) that binds to the fiber knob and prevents binding of the knob domain to the natural attachment receptor CAR (see FIG. 1). This central DARPin is then fused C-terminally to the trimerizing phage protein SHP, which mediates highly stable complex formation of three knob-binding DARPins and the fiber knob. N-terminally, the central DARPin is connected via a flexible linker to a second, exchangeable DARPin, that can be engineered to bind to any cell surface receptor of choice. Importantly, this modular DARPin adapter does not involve genetic fusion to the fiber and can hence be applied to any HAdV-C5-derived vector. In addition to the knob-binding retargeting adapter, a hexon-binding protein shield that extensively covers the surface of HAdV-C5 (Nat Commun (2018) 9:450) was developed. As a result, unspecific capsid interactions are even further decreased, but more importantly, shielding of the virion against the immune system, another major limitation to systemic viral vector delivery, can be achieved.

By applying tumor cell marker-specific DARPin adapters and the hexon-binding shield to a HVR7-mutated HAdV-C5, efficient in vivo transduction of tumor cells could be achieved, characterized by a highly reduced viral off-targeting. The thus improved vector specificity reinforced the idea of installing a local ‘biofactory’ of secreted cancer therapeutics, achieved through tumor cell-targeted viral gene delivery. However, this autocrine delivery approach might bring the disadvantage that the secretion of cancer therapeutics would be limited by the lifetime of the transduced tumor cells. Herein presented is a paracrine delivery system which is able to target the vector to FAP-positive cells, such as cancer cells, non-cancerous stromal cells in the tumor microenvironment (TME) or fibroblasts. Such an alternative approach would enable extended and sustained treatment opportunities. Furthermore, such a targeting strategy would be broader applicable and could also be used to treat a variety of cancer types, since the delivery can be tumor cell-independent, and hence independent of the existence of specific tumor cell markers. This independence additionally renders viral retargeting less susceptible to genetic changes that are observed in cancer cells, but not in the genetically more stable stromal cells. Finally, the possibility of also targeting the vector to stromal cells could be beneficial regarding the spatial distribution of the cellular components in the TME, especially in stroma-rich tumors. Stromalcell-targeted adenoviral vectors as described in the present disclosure significantly broaden treatment opportunities and help advance adenoviral therapeutic gene delivery.

To achieve this effect, the inventors use designed ankyrin repeat domains targeting FAP that are built into adapter molecules, which help the vector to effectively transduce FAP-positive cells, such as cancer cells, non-cancerous cells of the tumor microenvironment or fibroblasts. Adenoviruses armed with such adapter molecules can thus be used to deliver cargo into the tumor microenvironment.

Int. J. Mol. Sci (2021) 22, 8355 describes a system in which adeno-associated vectors (AAVs) are engineered in that a short linear epitope of PCSK9 is inserted into surface loops of the VP capsid protein. A bispecific antibody with one specificity for said PCSK9 epitope and one specificity for FAP is then used to direct the AAV's to fibroblasts. In Oncotarget (2017) 8, 76468, an oncolytic adenovirus was engineered in that short peptides targeting FAP where integrated into the fiber protein, thereby generating specificity for fibroblasts. Certain anti-FAP DARPins are disclosed in WO2020/245173. Certain multispecific polypeptides comprising anti-FAP DARPins are disclosed in Cancer Research, Annual Meeting of the AACR Vol. 78, No. 13, Suppl. S, 30 Jun. 2018, page 3752, DOI: 10.1158/1538-7445.AM2018-3752 and Eur J Cancer, 30th EORTC-NCI-AACR Symposium, vol. 103, Suppl. 1 31, page E78. The system disclosed in the present invention offers several advantages, such as the possibility to deliver cargo into cells of the tumor microenvironment in high-capacity vehicles.

### SUMMARY OF THE INVENTION

The present disclosure relates to a recombinant protein capable of mediating the transduction of FAP-positive cells comprising
a) a first designed ankyrin repeat domain specifically binding to FAP,
b) a second designed ankyrin repeat domain specifically binding to the knob of an adenovirus, and
c) a trimerization domain.

The present disclosure relates to a recombinant protein capable of mediating the adenoviral transduction of FAP-positive cells comprising
a) a first designed ankyrin repeat domain specifically binding to FAP,
b) a second designed ankyrin repeat domain specifically binding to the knob of an adenovirus, and
c) a trimerization domain.

In other embodiments, the present disclosure relates to recombinant proteins comprising
a) a first designed ankyrin repeat domain specifically binding to FAP,
b) a second designed ankyrin repeat domain specifically binding to the knob of an adenovirus, and
c) a trimerization domain. Said recombinant proteins are capable of mediating the transduction of FAP-positive cells, preferably wherein said FAP-positive cells are cancer cells, non-cancerous cells of the tumor microenvironment or fibroblasts.

The present disclosure also relates to aforementioned recombinant proteins, wherein the first designed ankyrin repeat domain of said recombinant proteins comprises an amino acid sequence of any one of SEQ ID Nos. 5-28, preferably an amino acid sequence selected from any one of SEQ ID Nos. 6, 7, 8, 9, 11, 13, 16, 22 and 23, more preferably an amino acid sequence selected from any one of SEQ ID Nos. 6, 7, 9 and 22.

The present disclosure also relates to aforementioned recombinant proteins, wherein said first designed ankyrin repeat domain is cross-reactive with mouse FAP, preferably wherein said first designed ankyrin repeat domain comprises the amino acid sequence of SEQ ID No. 9.

In certain embodiments, said recombinant protein comprises from the N- to the C-terminus said first designed ankyrin repeat domain specifically binding to FAP, said second designed ankyrin repeat domain specifically binding to the knob of an adenovirus and said trimerization domain.

In certain embodiments, said designed ankyrin repeat domain that specifically binds to the knob of an adenovirus comprises the amino acid sequence of SEQ ID No. 2.

In certain embodiments, said trimerization domain is or is derived from the capsid protein SHP of lambdoid phage 21. In certain embodiments, said trimerization domain comprises the amino acid sequence of SEQ ID No. 1.

The present disclosure also relates to nucleic acids encoding aforementioned recombinant proteins.

The present disclosure also relates to trimeric proteins comprising three of aforementioned recombinant proteins.

The present disclosure also relates to a recombinant adenovirus displaying aforementioned recombinant proteins or trimeric protein.

In certain embodiments, said adenovirus is an adenovirus of serotype 5 or an adenovirus comprising a knob of an adenovirus of serotype 5.

The present disclosure also relates to the use of aforementioned recombinant adenoviruses, recombinant proteins or trimeric proteins for the transduction of FAP-positive cells. In certain embodiments said FAP-positive cells are cancer cells, non-cancerous cells of the tumor microenvironment or fibroblasts. In certain embodiments aforementioned use is the use in medicine.

The present disclosure also relates to recombinant proteins comprising an ankyrin repeat domain specifically binding to FAP, wherein said ankyrin repeat domain comprises an amino acid sequence selected from the group consisting of SEQ ID No. 5-28, preferably an amino acid sequence selected from any one of SEQ ID Nos. 6, 7, 8, 9, 11, 13, 16, 22 and 23, more preferably an amino acid sequence selected from any one of SEQ ID Nos. 6, 7, 9 and 22.

### FIGURE LEGENDS

FIG. 1: Adenoviral capsid proteins and DARPin-based adapter module for HAdV-C5 retargeting. Shown on the left are the three major capsid proteins of HAdV-C5: Hexon, penton, and fiber including fiber knob. Shown on the right is the DARPin-based retargeting adapter: A central knob-binding DARPin is C-terminally fused to the phage SHP protein, and N-terminally to a retargeting DARPin specific for the target of choice. The SHP protein mediates trimerization and formation of a highly stable adapter-knob complex (PNAS (2013) 110 (10), E869-E877).

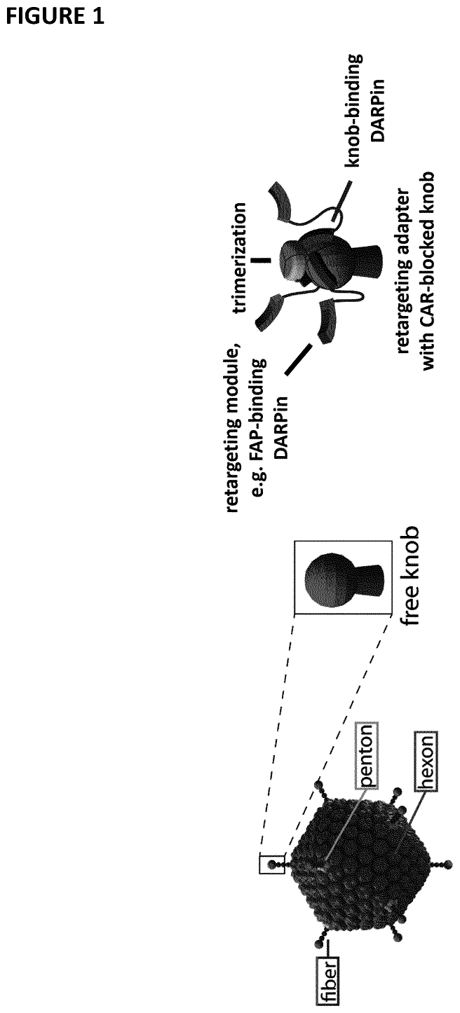

FIG. 2 shows the transduction efficiency of targeted (FAP-AdV), non-binding (E3_5 AdV) and untargeted (Untargeted AdV) vectors in a human fibrosarcoma cell line. Targeted vectors showed a significantly increased transduction efficiency in cells stably transfected with human FAP.

FIG. 3 shows the transduction efficiency of targeted (FAP-AdV) and untargeted (Untargeted AdV) vectors in the human embryonic fibroblast cell line Detroit 551. Transduction efficiencies were measured after 4 hours (top), 24 hours (middle) and 48 hours (bottom). Targeted vectors are shown on the left, untargeted vectors on the right.

FIG. 4 shows the transduction efficiency of targeted (FAP-AdV), non-binding (E3_5 AdV) and untargeted (Untargeted AdV) vectors in a mouse embryonic fibroblast cell line. Targeted vectors showed a significantly increased transduction efficiency in fibroblasts beforehand engineered to express murine FAP.

FIG. 5 shows the transduction efficiency of targeted (FAP-AdV) and untargeted (Untargeted AdV) vectors in vivo in tumor-bearing mice. Targeted vectors showed a significantly increased transduction efficiency in FAP⁺ fibroblasts.

FIG. 6 shows that FAP-specific adapters can be used to efficiently deliver functional anti-cancer therapeutics encoded by an adenoviral vector to the tumor microenvironment. Arrows indicate time points of injection for the corresponding treatment. Data points represent mean±SD. Statistic: Unpaired t-test; *p<0.05, **p<0.005, ***p<0.0005.

FIG. 7 shows the transduction efficiency of targeted (#3-6, #8, #10, #13, #20-21), non-binding (E3_5) and untargeted (Naked WT) vectors in a human fibrosarcoma cell line. Several adapters mediated a significantly increased adenoviral transduction efficiency in cells stably transfected with human FAP (FIG. 7A), but for some adapters the transduction was unspecific (FIG. 7B). Specific transduction was observed with vectors carrying adapters #3 (SEQ ID No. 6), #4 (SEQ ID No. 7), #6 (SEQ ID No. 9) and #20 (SEQ ID No. 22).

### DEFINITIONS

The term “recombinant” as used in recombinant protein, recombinant protein domain and the like, means that said polypeptides are produced by the use of recombinant DNA technologies well known by the practitioner skilled in the relevant art. For example, a recombinant DNA molecule (e.g. produced by gene synthesis) encoding a polypeptide can be cloned into a bacterial expression plasmid (e.g. pQE30, Qiagen). When such a constructed recombinant expression plasmid is inserted into a host cell (e.g. E. coli), this host cell can produce the polypeptide encoded by this recombinant DNA. The correspondingly produced polypeptide is called a recombinant polypeptide.

The term “protein” as used herein refers to a polypeptide, wherein at least part of the polypeptide has, or is able to, acquire a defined three-dimensional arrangement by forming secondary, tertiary, or quaternary structures within and/or between its polypeptide chain(s). If a protein comprises two or more polypeptides, the individual polypeptide chains may be linked non-covalently or covalently, e.g. by a disulfide bond between two polypeptides.

A part of a protein, which individually has, or is able to acquire a defined three-dimensional arrangement by forming secondary or tertiary structures, is termed “protein domain” or “domain”. Such protein domains are well known to the practitioner skilled in the art.

The term “polypeptide” as used herein refers to a molecule consisting of one or more chains of multiple, i.e. two or more, amino acids linked via peptide bonds. A polypeptide typically consists of more than twenty amino acids linked via peptide bonds.

The term “peptide” as used herein refers to as used herein refers to a molecule consisting of one or more chains of multiple, i.e. two or more, amino acids linked via peptide bonds. A peptide typically consists of not more than twenty amino acids linked via peptide bonds.

The terms “designed ankyrin repeat protein”, “designed ankyrin repeat domain” and “DARPin” as used herein refer to artificial polypeptides, comprising several ankyrin repeat motifs. These adjacent ankyrin repeat motifs provide a rigid interface arising from typically several adjacent helices and β-tums. DARPins usually carry two three repeats corresponding to an artificial consensus sequence, wherein six positions per repeat are randomized, flanked by two capping repeats with a hydrophilic surface (Curr Opin Chem Biol (2009) 13:245-55; WO 02/20565).

The term “antibody” as used herein refers to a protein comprising at least two heavy (H) chains and two light (L) chains inter-connected by disulfide bonds, which interacts with an antigen. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR).

Each VH and VL is composed of three CDRs and four FR's arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. The term “antibody” includes for example, monoclonal antibodies, human antibodies, humanized antibodies, camelised antibodies and chimeric antibodies. The antibodies can be of any isotype (e.g., IgG, IgE, IgM, IgD, IgA and IgY), class (e.g., IgG1, IgG2, IgG3, IgG4, IgA1 and IgA2) or subclass. Both the light and heavy chains are divided into regions of structural and functional homology.

The term “antibody fragment” as used herein refers to one or more portions of an antibody that retain the ability to specifically interact with (e.g., by binding, steric hindrance, stabilizing spatial distribution) an antigen. Examples of binding fragments include, but are not limited to, a Fab fragment, a monovalent fragment consisting of the VL, VH, CL and CH1 domains; a F (ab) 2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., (1989) Nature 341:544-546), which consists of a VH domain; and an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as “single chain variable fragment”, “single chain Fv” or “scFv”; see e.g., Bird et al., (1988) Science 242:423-426; and Huston et al., (1988) Proc. Natl. Acad. Sci. 85:5879-5883). Such single chain antibodies are also intended to be encompassed within the term “antibody fragment”. These antibody fragments are obtained using conventional techniques known to those of skill in the art, and the fragments are screened for utility in the same manner as are intact antibodies. Antibody fragments can also be incorporated into single domain antibodies, maxibodies, minibodies, intrabodies, diabodies, triabodies, tetrabodies, v-NAR and bis-scFv (see, e.g., Hollinger and Hudson, (2005) Nature Biotechnology 23:1 126-1 136). Antibody fragments can be grafted into scaffolds based on polypeptides such as Fibronectin type III (Fn3) (see U.S. Pat. No. 6,703,199, which describes fibronectin polypeptide monobodies). Antibody fragments can be incorporated into single chain molecules comprising a pair of tandem Fv segments (VH-CH1-VH-CH1) which, together with complementary light chain polypeptides, form a pair of antigen-binding sites (Zapata et al., (1995) Protein Eng. 8:1057-1062; and U.S. Pat. No. 5,641,870).

The term “immunoglobulin” as used herein refers to any polypeptide or fragment thereof from the class of polypeptides known to the skilled person under this designation and comprising at least one antigen binding site. Preferably, the immunoglobulin is a soluble immunoglobulin from any of the classes IgA, IgD, IgE, IgG, or IgM, or a fragment comprising at least one antigen binding site derived thereof. Also comprised as immunoglobulins of the present invention are a bispecific immunoglobulin, a synthetic immunoglobulin, an immunoglobulin fragment, such as Fab, Fv or scFv fragments etc., a single chain immunoglobulin, and a nanobody. Further included are chemically modified derivatives of any of the aforesaid, e.g. PEGylated derivatives, as well as fusion proteins comprising any of the aforesaid immunoglobulins and fragments thereof. The immunoglobulin may be a human or humanized immunoglobulin, a primatized, or a chimerized immunoglobulin or a fragment thereof as specified above. Preferably, the immunoglobulin of the present invention is a polyclonal or a monoclonal immunoglobulin, more preferably a monoclonal immunoglobulin or a fragment thereof as specified above.

The terms “binds”, “is specific” and “specifically binds” as used herein refers to a molecule, for example an antibody or an antibody fragment, which recognizes a specific antigen, but does not substantially recognize or bind other molecules in a sample. An antibody that specifically binds to an antigen from one species may also bind to that antigen from one or more further species. Such cross-species reactivity does not itself alter the classification of an antibody as specific.

The terms “cross-reactive” and “cross reactive” as used herein refer to a property of a binding protein or binding domain, such as a DARPin, to bind to more than one antigen of a similar type or class, such as an ortholog of an antigen. For example such binding protein may bind to the human and the non-human primate ortholog of the same antigen or to the human and the rodent ortholog of the same antigen. In preferred embodiments of the present disclosure the binding protein may bind to the human and the mouse ortholog of the same antigen.

The term “nucleic acid” as used herein refers to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The term “vector” as used herein means a construct, which is capable of delivering, and usually expressing or regulating expression of, one or more gene(s) or nucleic acid(s) of interest in a host cell. Examples of vectors include, but are not limited to, viral vectors, naked DNA or RNA expression vectors, plasmid, cosmid, or phage vectors, DNA or RNA expression vectors associated with cationic condensing agents, and DNA or RNA expression vectors encapsulated in liposomes.

The term “linker” as used herein refers a molecule or macromolecule serving to connect different moieties or domains of a peptide or a polypeptide or, a protein/polypeptide domain and a non-protein/non-polypeptide moiety. Linkers can be of different nature. Different domains or modules within proteins can be linked via peptide linkers. Linkers can also be generated chemically, for example to link small organic molecules or peptides to a protein.

The term “flexible linker” as used herein refers to a peptide linker linking two different domains or modules of a protein and providing a certain degree of flexibility. Preferably, the flexible linker is hydrophilic and does not interacting with other surfaces. Commonly used flexible linkers are glycine-serine linkers (Biochemistry 56 (50): 6565-6574 (2017)). Glycine and serine are flexible and the adjacent protein domains are free to move relative to one another. Such flexible linkers are referred to herein as “glycine-serine linkers”. Other amino acids commonly used in respective linkers are proline, asparagine and threonine. Often the linker contains several repeats of a sequence of amino acids. A flexible linker used in the present disclosure is a (Gly₄Ser)₄-linker, i.e. a linker containing four repeats of the sequence glycine-glycine-glycine-glycine-serine. Other linkers that could be used in accordance with the present disclosure include but are not limited to PAS linkers, i.e. linkers containing repeats of the sequence proline-alanine-serine (Protein Eng Des Sel (2013) 26, 489-501 and charged linkers.

The term “short linker” as used herein refers to a peptide linker linking two different domains or modules of a protein and which is no longer than four, preferably no longer than three amino acids long. More preferably the short linker is no longer than two amino acids long. Alternatively the short linker is only one amino acid long. Alternatively the short linker is a single glycine residue.

The term “amino acid mutation” refers to amino acid substitutions, deletions, insertions, and modifications, as well as combinations thereof. Amino acid sequence deletions and insertions include N- and/or C-terminal deletions and insertions of amino acid residues. Particular amino acid mutations are amino acid substitutions. Amino acid substitutions include replacement by non-naturally occurring amino acids or by naturally occurring amino acid derivatives of the twenty standard amino acids. Amino acid mutations can be generated using genetic or chemical methods well known in the art. Genetic methods may include site-directed mutagenesis, PCR, gene synthesis and the like. It is contemplated that methods of altering the side chain group of an amino acid residue by methods other than genetic engineering, such as chemical modification, may also be useful.

The term “variant” as used herein refers to a polypeptide that differs from a reference polypeptide by one or more amino acid mutation or modifications.

The term “host cell” as used herein refers to any kind of cellular system which can be engineered to generate molecules according to the present disclosure. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

Host cells according to the present disclosure can be a “eukaryotic host cell” and include yeast and mammalian cells, including murine cells and from other rodents, preferably vertebrate cells such as those from a mouse, rat, monkey or human cell line, for example HKB11 cells, PERC.6 cells, HEL293T cells, CHO cells or any type of HEK cells, such as HEK293 cells or HEK 993 cells. Also, suspension cell lines like CHO-S or HEK993 cells, or insect cell cultures like Sf9 cells may be used.

Host cells according to the present disclosure can also be “prokaryotic cell” and include bacterial cells, such Escherichia coli. Certain strains of Escherichia coli may be particularly useful for expression of the molecules of the present disclosure, such as Escherichia coli strain DH5 (available from Bethesda Research Laboratories, Inc., Bethesda, Md/US).

The molecules of the present disclosure form trimers that are highly stable. Each monomer contains a domain responsible for the formation of trimers which is referred to herein as “trimerization domain”. A preferred trimerization domain is the capsid protein SHP of lambdoid phage 21 (J Mol Biol; 344 (1): 179-93; PNAS 110 (10): E869-77 (2013)). SHP of lambdoid phage 21 has the following amino acid sequence:

(SEQ ID NO. 1)
VRIFAGNDPAHTATGSSGISSPTPALTPLMLDEATGKLVVWDGQKAGSA
VGILVLPLEGTETALTYYKSGTFATEAIHWPESVDEHKKANAFAGSALS
HAALP

The term “stable trimer” or “trimeric adapter” as used herein refers to a protein trimer by protein monomers comprising a trimerization domain, and wherein said trimer exhibits a stability which is higher than other, conventional protein trimers. For example, a stable trimer has a higher functional stability, a higher kinetic stability, or a higher high life for unfolding than other protein trimers. An example of a stable trimer is a trimer formed by monomers comprising the trimerization domain of the capsid protein SHP of lambdoid phage 21.

The term “derived from” in the context of an amino acid sequence refers to an amino acid sequence that is different to an original amino acid sequence, but maintains the function or activity of the original amino acid sequence. Such amino acid sequence may comprise an amino acid mutation as compared to the original amino acid sequence or may be a variant of the original amino acid sequence.

The term “adenovirus” as used herein refers to any adenovirus, i.e. to human and non-human serotypes. The human isolates are classified into species A-G. A preferred adenovirus of the present disclosure is adenovirus serotype 5 (“HAdV-C5”). HAdV-C5 includes modified version of the virus, such as replication-deficient HAdV-C5 version, e.g. containing an E1/E3 deletion and/or one or more of the 4 mutations in the HVR7 (1421G, T423N, E424S and L426Y) (Nat. Commun. 9, 450 (2018)).

The terms “CAR” and “CXADR” as used herein refers to coxsackievirus and adenovirus receptor (UniProt: P78310). CAR is a type I membrane receptor for coxsackie viruses and adenoviruses.

The term “knob” as used herein refers to a knob on the end of the adenovirus fiber (e.g. GenBank: AAP31231.1) that binds to the cellular receptor. The knob of adenovirus serotype 5 binds to CAR. Some adenoviruses carry mutations in the gene encoding the knob protein. Adenoviruses having a four-amino acid deletion within the FG loop of the knob (ATAYT mutation) show a decreased ability of the mutated knob to bind to CAR (Science, 286:1568-1571 (1999); J Mol Biol 405 (2): 410-426). Adenoviruses carrying four amino acid mutations in the hypervariable region 7 (HVR7 mutation) show a strongly reduced binding to blood coagulation factor X (Nat Commun (2018) 9:450).

The molecules of the present invention contain a designed ankyrin repeat domain that binds to the knob of an adenovirus. A preferred designed ankyrin repeat domain that binds to a knob is DARPin 1D3. Another preferred designed ankyrin repeat domain that binds to a knob is DARPin 1D3nc, a derivative of 1D3nc containing a stabilized C-cap. DARPin 1D3 has the following amino acid sequence:

(SEQ ID NO. 2)
RSDLGKKLLEAARAGQDDEVRILMANGADANAYDHYGRTPLHMAAAVGH
LEIVEVLLRNGADVNAVDTNGTTPLHLAASLGHLEIVEVLLKYGADVNA
KDATGITPLYLAAYWGHLEIVEVLLKHGADVNAQDKFGKTPFDLAIDNG
NEDIAEVLQG

The term “FAP” as used herein refers to a homodimeric integral membrane gelatinase belonging to the serine protease family. It is also known as fibroblast activation protein alpha, seprase, DPPIV, SIMP or FAPA and has the UniProt ID Q12884. FAP is selectively expressed on reactive stromal fibroblasts, such as cancer-associated fibroblasts, and some melanomas. FAP is found in more than 90% of all epithelial carcinomas, including breast, ovarian, lung, bladder, and colorectal carcinoma. Human FAP has the following amino acid sequence:

(SEQ ID No. 3)
MKTWVKIVFGVATSAVLALLVMCIVLRPSRVHNSEENTMRALTLKDILNGTFSYKTFFPNWISGQE
YLHQSADNNIVLYNIETGQSYTILSNRTMKSVNASNYGLSPDRQFVYLESDYSKLWRYSYTATYYI
YDLSNGEFVRGNELPRPIQYLCWSPVGSKLAYVYQNNIYLKQRPGDPPFQITENGRENKIFNGIPD
WVYEEEMLATKYALWWSPNGKFLAYAEFNDTDIPVIAYSYYGDEQYPRTINIPYPKAGAKNPVVRI
FIIDTTYPAYVGPQEVPVPAMIASSDYYFSWLTWVTDERVCLQWLKRVQNVSVLSICDFREDWQTW
DCPKTQEHIEESRTGWAGGFFVSTPVFSYDAISYYKIFSDKDGYKHIHYIKDTVENAIQITSGKWE
AINIFRVTQDSLFYSSNEFEEYPGRRNIYRISIGSYPPSKKCVTCHLRKERCQYYTASFSDYAKYY
ALVCYGPGIPISTLHDGRTDQEIKILEENKELENALKNIQLPKEEIKKLEVDEITLWYKMILPPQF
DRSKKYPLLIQVYGGPCSQSVRSVFAVNWISYLASKEGMVIALVDGRGTAFQGDKLLYAVYRKLGV
YEVEDQITAVRKFIEMGFIDEKRIAIWGWSYGGYVSSLALASGTGLFKCGIAVAPVSSWEYYASVY
TERFMGLPTKDDNLEHYKNSTVMARAEYFRNVDYLLIHGTADDNVHFQNSAQIAKALVNAQVDFQA
MWYSDQNHGLSGLSTNHLYTHMTHFLKQCFSLSD

Mouse FAP has the following amino acid sequence:

(SEQ ID NO. 4)
MKTWLKTVFGVTTLAALALVVICIVLRPSRVYKPEGNTKRALTLKDILNGTFSYKTYFPNWISEQE
YLHQSEDDNIVFYNIETRESYIILSNSTMKSVNATDYGLSPDRQFVYLESDYSKLWRYSYTATYYI
YDLQNGEFVRGYELPRPIQYLCWSPVGSKLAYVYQNNIYLKQRPGDPPFQITYTGRENRIFNGIPD
WVYEEEMLATKYALWWSPDGKFLAYVEENDSDIPIIAYSYYGDGQYPRTINIPYPKAGAKNPVVRV
FIVDTTYPHHVGPMEVPVPEMIASSDYYFSWLTWVSSERVCLQWLKRVQNVSVLSICDFREDWHAW
ECPKNQEHVEESRTGWAGGFFVSTPAFSQDATSYYKIFSDKDGYKHIHYIKDTVENAIQITSGKWE
AIYIFRVTQDSLFYSSNEFEGYPGRRNIYRISIGNSPPSKKCVTCHLRKERCQYYTASFSYKAKYY
ALVCYGPGLPISTLHDGRTDQEIQVLEENKELENSLRNIQLPKVEIKKLKDGGLTFWYKMILPPQF
DRSKKYPLLIQVYGGPCSQSVKSVFAVNWITYLASKEGIVIALVDGRGTAFQGDKFLHAVYRKLGV
YEVEDQLTAVRKFIEMGFIDEERIAIWGWSYGGYVSSLALASGTGLFKCGIAVAPVSSWEYYASIY
SERFMGLPTKDDNLEHYKNSTVMARAEYFRNVDYLLIHGTADDNVHFQNSAQIAKALVNAQVDFQA
MWYSDQNHGISSGRSQNHLYTHMTHFLKQCFSLSD

The term “FAP-positive cell” as used herein refers to cells expressing human FAP. Various cells have been described to express human FAP, including cancer cells, non-cancerous cells of the tumor microenvironment and fibroblasts. FAP is expressed on reactive stromal fibroblasts of more than 90% of epithelial malignancies (primary and metastatic), including lung, colorectal, prostate, ovarian, and breast carcinomas (Front Oncol (2021) 11:648187).

The term “displaying” as used herein refers to the presentation of a polypeptide on the outside of an entity, such as an adenovirus. The polypeptides so presented on the entity may be covalently or non-covalently attached to such entity. In the context of the present disclosure, adapter molecules are recombinantly expressed and displayed on adenoviruses. This can be accomplished via a binding moiety or a scaffold, such as a designed ankyrin repeat domain that binds to the knob of an adenovirus. Alternatively, moiety or scaffold can also be genetically fused to an adenoviral protein, such as the hexon.

### Embodiments of the Invention

Disclosed herein is an adenoviral-based system for the transduction of human fibroblasts. The system can be used in medicine, such as diseases and disorders associated with fibroblasts, including cancer. The adenoviral-based systems allows the paracrine delivery of therapeutic biomolecules by transducing non-cancerous stromal cells in the tumor microenvironment (TME). Cargo, such as nucleic acids, in particular nucleic acids encoding therapeutically active or therapeutically helpful proteins and peptides, can be delivered to fibroblasts to then exert their function in the tumor microenvironment.

Specificity of the adenoviruses is conferred by adapter molecules as described in the present disclosure. The specificity of the system and the correlating transduction efficiency is considerably higher than the systems known in the art. The system is functional with adenoviruses of any kind, including first-generation viruses, as well as high-capacity, helper virus-dependent adenoviral systems and replication-competent adenoviruses. The system is also functional with other viruses, e.g. viruses that are engineered to carry a knob of an adenovirus of serotype 5.

In certain embodiments, the present disclosure relates to a recombinant protein capable of mediating the transduction of FAP-positive cells comprising
a) a first designed ankyrin repeat domain specifically binding to FAP,
b) a second designed ankyrin repeat domain specifically binding to the knob of an adenovirus, and
c) a trimerization domain.

In certain embodiments, the present disclosure relates to a recombinant protein comprising
a) a first designed ankyrin repeat domain specifically binding to FAP,
b) a second designed ankyrin repeat domain specifically binding to the knob of an adenovirus, and
c) a trimerization domain.

In certain embodiments, the present disclosure relates to a recombinant protein comprising
a) a first designed ankyrin repeat domain specifically binding to FAP,
b) a second designed ankyrin repeat domain specifically binding to the knob of an adenovirus, and
c) a trimerization domain, wherein said first designed ankyrin repeat domain is capable of mediating the transduction of human fibroblasts.

The system described in the present disclosure greatly enhances the transduction rate of adenoviruses for human fibroblasts. Adenoviruses equipped with the adapter molecules of the present disclosure have a significantly increased transduction rate as compared to untargeted adenoviruses and as compared to adenoviruses comprising other non-specific adapter molecules, while at the same time offering the possibility to deliver cargo into fibroblasts, and hence the tumor microenvironment. The measurement of transduction efficiency will be a standard procedure for a person skilled in the art of flow cytometry or fluorescence microscopy, and can for example be performed as described in the Examples of the present disclosure.

The system described herein makes use of designed ankyrin repeat domains that specifically bind to FAP. In certain embodiments the present disclosure relates to a designed ankyrin repeat domain comprising an amino acid sequence selected from any one of SEQ ID Nos. 5-28. Preferably said designed ankyrin repeat domain comprises an amino acid sequence selected from any one of SEQ ID Nos. 6, 7, 8, 9, 11, 13, 16, 22 and 23. More preferably said designed ankyrin repeat domain comprises an amino acid sequence selected from any one of SEQ ID Nos. 6, 7, 9 and 22.

TABLE 1

| Designed ankyrin | | SEQ ID |
|---|---|---|
| repeat domain | Amino acid sequence | NO. |
| #1 | DLGKKLLDAARAGQDDEVRILMANGADVNAMDVWGVTPLHLAAW | 5 |
| | HRHLEIVEVLLKTGADVNANDDSGFTPLHLAAHWGHLEIVEVLL | |
| | KHGADVNAQDKFGKTPFDLAIDNGNEDIAEVLQ | |
| #3 | DLGKKLLEAARAGQDDEVRILMANGADVNAQDMTGKTPLHLAAR | 6 |
| | EGHLEIVEVLLKTGADVNAKDLWGVTPLHLAAFSGHLEIVEVLL | |
| | KTGADVNAWDWEGKTPLHLAAVKGHPEIVEVLLKHGADVNAQDK | |
| | FGKTPFDLAIDNGNEDIAEVLQ | |
| #4 | DLGKKLLDAASVGQDDEVRILMANGADVNASDTYGRTPLHVAAQ | 7 |
| | HGHLEIVDVLLAHGADVNASNAYGSTPLHVAAWTGHLEIVDVLL | |
| | AYGADVNASNSQGNTPLHAAAATGHLEIVEVLLKHGADVNAQDK | |
| | FGKTPFDLAIDNGNEDIAEVLQ | |
| #5 | DLGKKLLEAARAGQDDEVRILMANGADVNAEDHVGRTPLHLAAA | 8 |
| | HGHLEIVEVLLKTGADVNARDRHGITPLHLAAWSGHLEIVEVLL | |
| | KTGADVNASDKQGNTPLHLAAYRGHLEIVEVLLEAGADVNAQDK | |
| | FGKTPFDLAIDNGNEDIAEVLQ | |
| #6 | DLGKKLLEAARAGQDDEVRILMANGADVNASDNWGRTPLHVAAQ | 9 |
| | RGHLEIVDVLLAHGADDNASNRWGSTPLHVAAYTGHLEIVDVLL | |
| | ANGADVNASTANGETPLHAAAHAGHLEIVDVLLKHGADVNAQDK | |
| | FGKTPFDLAIDNGNEDIAEVLQ | |
| #7 | DLGKKLLXAARAGQDDEVRILMANGADVNAXDXFGXTPLHLAAW | 10 |
| | XGHLEIVEVLLKAGADVNAXDXWGNTPLHLAAHRGHLEIVEVLL | |
| | KHGADVNAQDKFGKTPFDLAIDNGNEDIAEVLQ | |
| #8 | DLGKKLLEAARAGQDDEVRILMANGADVNAADEYGITPLHLAAF | 11 |
| | FGHLEIVEVLLKTGADVNADDAWGSTPLHLAAYKGHLEIVEVLL | |
| | KHGADVNAQDKFGKTPFDLAIDNGNEDIAEVLQ | |
| #9 | DLGKKLLEAARAGQDDEVRILMANGADVNAXDWWGITPLHLAAX | 12 |
| | VGHLEIVEVLLKTGADVNABDKXGXTPLHLAAYXGHLEIVEVLL | |
| | XXGADVNAZDKFGKTPFDLAIDNGNEDIAEVLQ | |
| #10 | DLGKKLLEAARAGQDDEVRILMANGADVNAADDQGRTPLHLAAM | 13 |
| | HGHLEIVEVLLKTGADVNANDWFGITPLHLAAWWGHLEIVEVLL | |
| | KAGADVNAVDHSGDTPLHLAAHLGHLEIVEVLLKHGADVNAQDK | |
| | FGKTPFDLAIDNGNEDIAEVLQ | |
| #11 | DLVKKLLEAARAGQDDEVRILMANGADVNANDHFGITPLHLTAW | 14 |
| | WGHLEIVEVLLKAGADVNAVDHWGTTPLHLAAHQGHLEIVEVLL | |
| | KHGADVNAQDKFGKTAFDISIDNGNEDIAEVLQ | |
| #12 | DLGKKLLEAARAGQDDEVRILMANGADVNAQDQRGRTPLHLAAA | 15 |
| | WGHLEIVEVLLKTGADVNAADDMGHTPLHLAAAFGHLEIVEVLL | |
| | KHGADVNAQDKFGKTPFDLAIDNGNEDIAEVLQ | |
| #13 | DLGKKLLEAARAGQDDEVRILMANGADVNATDQHGRTPLHLAAH | 16 |
| | TGHLEIVEVLLKTGADVNAQDHQGITPLHLAAFKGHLEIVEVLL | |
| | KTGADVNAQDNWGYTPLHLAAARGHLEIVEVLLKAGADVNAQDK | |
| | FGKTPFDLAIDNGNEDIAEVLQ | |
| #15 | DLGKKLLEAARAGQDDEVRILMANGADVNAMDMWGITPLHLAAW | 17 |
| | AGHLEIVEVLLKTGADVNAFDQHGNTPLHLAAYRGHLEIVEVLL | |
| | KTGADVNAHDNWGTTPLHLTAHNGHLEIVEVLLKAGADVNAQDK | |
| | FGKTPFDLAIDNGNEDIAEVLQ | |
| #16 | DLGKKLLEAARAGQDDEVRILMANGADVNAKDRYGITPLHLAAY | 18 |
| | WGHLEIVEVLLKAGSDVNALDYHGSTPLHLAAWFGHLEIVEVLL | |
| | KHGADVNAQDKFGKTPFDLAIDNGNEDIAEVLQ | |
| #17 | DLGKKLLEAARAGQDDEVRILMANGADVNAADNYGITPLHLAAY | 19 |
| | RGHLEIVEVLLKTGADVNAHDNVGNTPLHLAAAKGHLEIVEVLL | |
| | KAGADVNADDVQGVTPLHLAASQGHLEIVEVLLKHGADVNAQDK | |
| | FGKTPFDLAIDNGNEDIAEVLQ | |
| #18 | DLGKKLLEAARAGQDDEVRILMANGADVNAVDHYGITPLHLAAF | 20 |
| | WGHLEIVEVLLKTGADVNAXDXWGWTPLHLAAVRGHLEIVEVLL | |
| | KHGADVNAQDKFGKTPFDLAIDNGNEDIAEVLQ | |
| #19 | DLGKKLLEAARAGQDDEVRILMANGADVNAXDWXGITPLHLAAF | 21 |
| | XGHLEIVEVLLKAGADVNAVDSDGSTPLHLAAYXGHLEIVEVLL | |
| | KHGADVNAQDKFGKTPFDLAIDNGNEDIAEVLQ | |
| #20 | DLGKKLLEAARAGQDDEVRILMANGADVNAVDYQGRTPLHLAAQ | 22 |
| | YGHLEIVEVLLKTGADVNAQDHHGITPLHLAAFWGHLEIVEVLL | |
| | KAGADVNAKDDQGNTPLHLAAWRGHLEIVEVLLKHGADVNAQDK | |
| | FGKTPFDLAIDNGNEDIAEVLQ | |
| #21 | DLGKKLLEAAELGQLDEVRILMANGADVNAMDQHGITPLHLAAW | 23 |
| | HGHLEIVEVLLKTGADVNAKDQDGSTPLHLAAYLGHLEIVEVLL | |
| | KTGADVNAFDKFGNTPLHLAAHRGHLEIVEVLLKHGADVNAQDK | |
| | FGKTPFDLAIDNGNEDIAEVLQ | |
| #22 | DLGKKLLEAARAGQDDEVRILMANGADVNASDWWGITPLHLAAY | 24 |
| | WGHLEIVEVLLKTGADVNAQDNQGITPLHLAATLGHLEIVEVLL | |
| | KHGADVNAQDKFGKTPFDLAIDNGNEDIAEVLQ | |
| #23 | DLGKKLLEAARYGQDDEVRILMANGADVNAEDQHGHTPLHLAAF | 25 |
| | SGHLEIVEVLLKTGADVNANDINGVTPLHLAANVGHLEIVEVLL | |
| | KHGADVNAQDKFGKTPFDLAAYLGNEDIAEVLQ | |
| #24 | DLGKKLLEAARAGQDDEVRILMANGADVNAIDVHGITPLHLAAY | 26 |
| | WGHLEIVEVLLKAGADVNAEDESGHTPLHLAAHWGHLEIVEVLL | |
| | KHGADVNAQDKFGKTPFDLAIDNGNEDIAEVLQ | |
| #25 | DLGKKLLEAARAGQDDEVRILMANGADVNATDWQGSTPLHLAAQ | 27 |
| | HGHLEIVEVLLKTGADVNAEDQNGITPLHLAAYWGHLEIVEVLL | |
| | KAGADVNAVDRQGNTPLHLAAVWGHLEIVEVLLKHGADVNAQDK | |
| | FGKTPFDLAIDNGNEDIAEVLQ | |
| #26 | DLGKKLLEAARAGQDDEVRILMANGADVNAIDWWGITPLHLAAL | 28 |
| | FGHLEIVEVLLKTGADVNAEDRLGFTPLHLAATTGHLEIVEVLL | |
| | KAGADVNAQDKFGKTPEDLAIDNGNEDIAEVLQ | |

In certain embodiments the present disclosure relates to a designed ankyrin repeat domain comprising an amino acid sequence of SEQ ID No. 5.

In certain embodiments the present disclosure relates to a designed ankyrin repeat domain comprising an amino acid sequence of SEQ ID No. 6.

In certain embodiments the present disclosure relates to a designed ankyrin repeat domain comprising an amino acid sequence of SEQ ID No. 7.

In certain embodiments the present disclosure relates to a designed ankyrin repeat domain comprising an amino acid sequence of SEQ ID No. 8.

In certain embodiments the present disclosure relates to a designed ankyrin repeat domain comprising an amino acid sequence of SEQ ID No. 9.

In certain embodiments the present disclosure relates to a designed ankyrin repeat domain comprising an amino acid sequence of SEQ ID No. 10.

In certain embodiments the present disclosure relates to a designed ankyrin repeat domain comprising an amino acid sequence of SEQ ID No. 11.

In certain embodiments the present disclosure relates to a designed ankyrin repeat domain comprising an amino acid sequence of SEQ ID No. 12.

In certain embodiments the present disclosure relates to a designed ankyrin repeat domain comprising an amino acid sequence of SEQ ID No. 13.

In certain embodiments the present disclosure relates to a designed ankyrin repeat domain comprising an amino acid sequence of SEQ ID No. 14.

In certain embodiments the present disclosure relates to a designed ankyrin repeat domain comprising an amino acid sequence of SEQ ID No. 15.

In certain embodiments the present disclosure relates to a designed ankyrin repeat domain comprising an amino acid sequence of SEQ ID No. 16.

In certain embodiments the present disclosure relates to a designed ankyrin repeat domain comprising an amino acid sequence of SEQ ID No. 17.

In certain embodiments the present disclosure relates to a designed ankyrin repeat domain comprising an amino acid sequence of SEQ ID No. 18.

In certain embodiments the present disclosure relates to a designed ankyrin repeat domain comprising an amino acid sequence of SEQ ID No. 19.

In certain embodiments the present disclosure relates to a designed ankyrin repeat domain comprising an amino acid sequence of SEQ ID No. 20.

In certain embodiments the present disclosure relates to a designed ankyrin repeat domain comprising an amino acid sequence of SEQ ID No. 21.

In certain embodiments the present disclosure relates to a designed ankyrin repeat domain comprising an amino acid sequence of SEQ ID No. 22.

In certain embodiments the present disclosure relates to a designed ankyrin repeat domain comprising an amino acid sequence of SEQ ID No. 23.

In certain embodiments the present disclosure relates to a designed ankyrin repeat domain comprising an amino acid sequence of SEQ ID No. 24.

In certain embodiments the present disclosure relates to a designed ankyrin repeat domain comprising an amino acid sequence of SEQ ID No. 25.

In certain embodiments the present disclosure relates to a designed ankyrin repeat domain comprising an amino acid sequence of SEQ ID No. 26.

In certain embodiments the present disclosure relates to a designed ankyrin repeat domain comprising an amino acid sequence of SEQ ID No. 27.

In certain embodiments the present disclosure relates to a designed ankyrin repeat domain comprising an amino acid sequence of SEQ ID No. 28. In certain embodiments the present disclosure relates to a designed ankyrin repeat domain comprising an amino acid sequence selected from any one of SEQ ID Nos. 5-28, wherein said designed ankyrin repeat domain is capable of mediating the transduction of FAP-positive cells. Preferably said designed ankyrin repeat domain comprises an amino acid sequence selected from any one of SEQ ID Nos. 6, 7, 8, 9, 11, 13, 16, 22 and 23. More preferably said designed ankyrin repeat domain comprises an amino acid sequence selected from any one of SEQ ID Nos. 6, 7, 9 and 22.

In certain embodiments the present disclosure relates to a designed ankyrin repeat domain comprising an amino acid sequence selected from any one of SEQ ID Nos. 5-28, wherein said designed ankyrin repeat domain is capable of mediating the transduction of FAP-positive cells, wherein said FAP-positive cells are cancer cells. Preferably said designed ankyrin repeat domain comprises an amino acid sequence selected from any one of SEQ ID Nos. 6, 7, 8, 9, 11, 13, 16, 22 and 23. More preferably said designed ankyrin repeat domain comprises an amino acid sequence selected from any one of SEQ ID Nos. 6, 7, 9 and 22.

In certain embodiments the present disclosure relates to a designed ankyrin repeat domain comprising an amino acid sequence selected from any one of SEQ ID Nos. 5-28, wherein said designed ankyrin repeat domain is capable of mediating the transduction of FAP-positive cells, wherein said FAP-positive cells are cells of the tumor microenvironment. Preferably said designed ankyrin repeat domain comprise an amino acid sequence selected from any one of SEQ ID Nos. 6, 7, 8, 9, 11, 13, 16, 22 and 23. More preferably said designed ankyrin repeat domain comprises an amino acid sequence selected from any one of SEQ ID Nos. 6, 7, 9 and 22.

In certain embodiments the present disclosure relates to a designed ankyrin repeat domain comprising an amino acid sequence selected from any one of SEQ ID Nos. 5-28, wherein said designed ankyrin repeat domain is capable of mediating the transduction of FAP-positive cells, wherein said FAP-positive cells are fibroblasts. Preferably said designed ankyrin repeat domain comprises an amino acid sequence selected from any one of SEQ ID Nos. 6, 7, 8, 9, 11, 13, 16, 22 and 23. More preferably said designed ankyrin repeat domain comprises an amino acid sequence selected from any one of SEQ ID Nos. 6, 7, 9 and 22.

In certain embodiments the present disclosure relates to a designed ankyrin repeat domain comprising an amino acid sequence selected from any one of SEQ ID Nos. 5-28, wherein said designed ankyrin repeat domain is capable of mediating the transduction of human fibroblasts. Preferably said designed ankyrin repeat domain comprises an amino acid sequence selected from any one of SEQ ID Nos. 6, 7, 8, 9, 11, 13, 16, 22 and 23. More preferably said designed ankyrin repeat domain comprises an amino acid sequence selected from any one of SEQ ID Nos. 6, 7, 9 and 22.

In certain embodiments, the present disclosure relates to a recombinant protein comprising
a) a first designed ankyrin repeat domain specifically binding to FAP,
b) a second designed ankyrin repeat domain specifically binding to the knob of an adenovirus, and
c) a trimerization domain,
wherein said first designed ankyrin repeat domain comprises an amino acid sequence selected from any one of SEQ ID Nos. 5-28. Preferably said designed ankyrin repeat domain comprise an amino acid sequence selected from any one of SEQ ID Nos. 6, 7, 8, 9, 11, 13, 16, 22 and 23. More preferably said designed ankyrin repeat domain comprises an amino acid sequence selected from any one of SEQ ID Nos. 6, 7, 9 and 22.

In certain embodiments, the present disclosure relates to a recombinant protein comprising
a) a first designed ankyrin repeat domain specifically binding to FAP,
b) a second designed ankyrin repeat domain specifically binding to the knob of an adenovirus, and
c) a trimerization domain,
wherein said first designed ankyrin repeat domain comprises an amino acid sequence selected from any one of SEQ ID Nos. 5-28, and
wherein said first designed ankyrin repeat domain is capable of mediating the transduction of FAP-positive cells. Said FAP-positive cells may be cancer cells, non-cancerous cells of the tumor microenvironment or fibroblasts. Preferably said designed ankyrin repeat domain comprise an amino acid sequence selected from any one of SEQ ID Nos. 6, 7, 8, 9, 11, 13, 16, 22 and 23. More preferably said designed ankyrin repeat domain comprises an amino acid sequence selected from any one of SEQ ID Nos. 6, 7, 9 and 22.

In certain embodiments, the present disclosure relates to a recombinant protein comprising
a) a first designed ankyrin repeat domain specifically binding to FAP,
b) a second designed ankyrin repeat domain specifically binding to the knob of an adenovirus, and
c) a trimerization domain,
wherein said first designed ankyrin repeat domain comprises an amino acid sequence selected from any one of SEQ ID Nos. 5-28, and
wherein said first designed ankyrin repeat domain is capable of mediating the transduction of human fibroblasts. Preferably said designed ankyrin repeat domain comprise an amino acid sequence selected from any one of SEQ ID Nos. 6, 7, 8, 9, 11, 13, 16, 22 and 23. More preferably said designed ankyrin repeat domain comprises an amino acid sequence selected from any one of SEQ ID Nos. 6, 7, 9 and 22.

In certain embodiments, the present disclosure relates to a designed ankyrin repeat domain specifically binding to human FAP. In certain embodiments, the present disclosure relates to a designed ankyrin repeat domain specifically binding to human FAP, wherein said designed ankyrin repeat domain is cross-reactive with mouse FAP. In certain embodiments, the present disclosure relates to a designed ankyrin repeat domain specifically binding to human FAP, wherein said designed ankyrin repeat domain comprises the amino acid sequence of SEQ ID No. 9, and wherein said designed ankyrin repeat domain is cross-reactive with mouse FAP.

In certain embodiments, the present disclosure relates to a recombinant protein comprising
a) a first designed ankyrin repeat domain specifically binding to human FAP,
b) a second designed ankyrin repeat domain specifically binding to the knob of an adenovirus, and
c) a trimerization domain.

In certain embodiments, the present disclosure relates to a recombinant protein comprising
a) a first designed ankyrin repeat domain specifically binding to human FAP,
b) a second designed ankyrin repeat domain specifically binding to the knob of an adenovirus, and
c) a trimerization domain, wherein said first designed ankyrin repeat domain is cross-reactive with mouse FAP.

In certain embodiments, the present disclosure relates to a recombinant protein comprising
a) a first designed ankyrin repeat domain specifically binding to human FAP,
b) a second designed ankyrin repeat domain specifically binding to the knob of an adenovirus, and
c) a trimerization domain, wherein said first designed ankyrin repeat domain comprises the amino acid sequence of SEQ ID No. 9, and wherein said first designed ankyrin repeat domain is cross-reactive with mouse FAP.

The recombinant proteins of the present disclosure comprise a first designed ankyrin repeat domain specifically binding to FAP, a second designed ankyrin repeat domain specifically binding to the knob of an adenovirus, and a trimerization domain. Said first designed ankyrin repeat domain specifically binding to FAP, said second designed ankyrin repeat domain specifically binding to the knob of an adenovirus and said trimerization domain may be fused to the other parts of the recombinant protein in any order.

In certain embodiments, the present disclosure relates to a recombinant protein comprising
a) a first designed ankyrin repeat domain specifically binding to FAP,
b) a second designed ankyrin repeat domain specifically binding to the knob of an adenovirus, and
c) a trimerization domain, wherein said recombinant protein comprises from the N- to the C-terminus said first designed ankyrin repeat domain specifically binding to FAP, said second designed ankyrin repeat domain specifically binding to the knob of an adenovirus and said trimerization domain.

In certain embodiments, the present disclosure relates to a recombinant protein comprising
a) a first designed ankyrin repeat domain specifically binding to FAP,
b) a second designed ankyrin repeat domain specifically binding to the knob of an adenovirus, and
c) a trimerization domain,
wherein said recombinant protein comprises from the N- to the C-terminus said first designed ankyrin repeat domain specifically binding to FAP, said second designed ankyrin repeat domain specifically binding to the knob of an adenovirus and said trimerization domain, and
wherein said first designed ankyrin repeat domain is capable of mediating the transduction of human fibroblasts.

In certain embodiments, the present disclosure relates to a recombinant protein comprising
a) a first designed ankyrin repeat domain specifically binding to FAP,
b) a second designed ankyrin repeat domain specifically binding to the knob of an adenovirus, and
c) a trimerization domain,
wherein said recombinant protein comprises from the N- to the C-terminus said first designed ankyrin repeat domain specifically binding to FAP, said second designed ankyrin repeat domain specifically binding to the knob of an adenovirus and said trimerization domain, and
wherein said first designed ankyrin repeat domain comprises an amino acid sequence selected from any one of SEQ ID Nos. 5-28. Preferably said designed ankyrin repeat domain comprise an amino acid sequence selected from any one of SEQ ID Nos. 6, 7, 8, 9, 11, 13, 16, 22 and 23. More preferably said designed ankyrin repeat domain comprises an amino acid sequence selected from any one of SEQ ID Nos. 6, 7, 9 and 22.

In certain embodiments, the present disclosure relates to a recombinant protein comprising
a) a first designed ankyrin repeat domain specifically binding to human FAP,
b) a second designed ankyrin repeat domain specifically binding to the knob of an adenovirus, and
c) a trimerization domain,
wherein said recombinant protein comprises from the N- to the C-terminus said first designed ankyrin repeat domain specifically binding to FAP, said second designed ankyrin repeat domain specifically binding to the knob of an adenovirus and said trimerization domain, and
wherein said first designed ankyrin repeat domain is cross-reactive with mouse FAP.

In certain embodiments, the present disclosure relates to a recombinant protein comprising
a) a first designed ankyrin repeat domain specifically binding to human FAP,
b) a second designed ankyrin repeat domain specifically binding to the knob of an adenovirus, and
c) a trimerization domain,
wherein said recombinant protein comprises from the N- to the C-terminus said first designed ankyrin repeat domain specifically binding to FAP, said second designed ankyrin repeat domain specifically binding to the knob of an adenovirus and said trimerization domain, and
wherein said first designed ankyrin repeat domain is cross-reactive with mouse FAP and comprises the amino acid sequence of SEQ ID No. 9.

The recombinant proteins of the present disclosure comprise a designed ankyrin repeat domain which binds to the knob of a virus or adenovirus. It will be appreciated that any designed ankyrin repeat domain with specificity for the knob of a virus or adenovirus may be used within the spirit of the present disclosure. Exemplified herein is a designed ankyrin repeat domain derived from DARPin 1D3 (Proc. Natl. Acad. Sci. 110, E869-E877 (2013)). DARPin 1D3 binds to the knob of an adenovirus and comprises the amino acid sequence of SEQ ID No. 2. Used herein is 1D3nc, a derivative of 1D3 containing a stabilized C-cap.

It will also be understood that also variants of DARPin 1D3 may be used within the spirit of the present disclosure. In other words, the amino acid sequence of such modified DARPin 1D3 does not need to be identical to that of amino acid sequence of SEQ ID No. 2, but may contain amino acids mutations, provided that the function of DARPin 1D3, i.e. binding to the knob of an adenovirus is preserved. Also DARPins different than 1D3, but having the same target specificity, may be used within the scope of the present disclosure. Such new DARPin may for example be selected in a new screening campaign. Also binding entities different than DARPins, i.e. binders based on a different scaffold, but having the same target specificity as 1D3 might be used.

Therefore, in certain embodiments, the present disclosure relates to a recombinant protein comprising a recombinant protein comprising
a) a first designed ankyrin repeat domain specifically binding to FAP,
b) a second designed ankyrin repeat domain specifically binding to the knob of an adenovirus, and
c) a trimerization domain,
wherein said designed ankyrin repeat domain which binds to the knob of an adenovirus is DARPin 1D3.

In certain embodiments, the present disclosure relates to a recombinant protein comprising a recombinant protein comprising
a) a first designed ankyrin repeat domain specifically binding to FAP,
b) a second designed ankyrin repeat domain specifically binding to the knob of an adenovirus, and
c) a trimerization domain,
wherein said designed ankyrin repeat domain which binds to the knob of an adenovirus is or is derived from DARPin 1D3.

In certain embodiments, the present disclosure relates to a recombinant protein comprising a recombinant protein comprising
a) a first designed ankyrin repeat domain specifically binding to FAP,
b) a second designed ankyrin repeat domain specifically binding to the knob of an adenovirus, and
c) a trimerization domain,
wherein said designed ankyrin repeat domain which binds to the knob of an adenovirus is a variant of DARPin 1D3.

In certain embodiments, the present disclosure relates to a recombinant protein comprising a recombinant protein comprising
a) a first designed ankyrin repeat domain specifically binding to FAP,
b) a second designed ankyrin repeat domain specifically binding to the knob of an adenovirus, and
c) a trimerization domain,
wherein said designed ankyrin repeat domain which binds to the knob of an adenovirus comprises the amino acid sequence of SEQ ID No. 2.

In certain embodiments, the present disclosure relates to a recombinant protein comprising a recombinant protein comprising
a) a first designed ankyrin repeat domain specifically binding to FAP,
b) a second designed ankyrin repeat domain specifically binding to the knob of an adenovirus, and
c) a trimerization domain,
wherein said designed ankyrin repeat domain which binds to the knob of an adenovirus comprises a variant of the amino acid sequence of SEQ ID No. 2.

In certain embodiments, the present disclosure relates to a recombinant protein comprising a recombinant protein comprising
a) a first designed ankyrin repeat domain specifically binding to FAP,
b) a second designed ankyrin repeat domain specifically binding to the knob of an adenovirus, and
c) a trimerization domain,
wherein said second designed ankyrin repeat domain which binds to the knob of an adenovirus comprises a variant of the amino acid sequence of SEQ ID No. 2, and
wherein said first designed ankyrin repeat domain comprises an amino acid sequence selected from any one of SEQ ID Nos. 5-28. Preferably said designed ankyrin repeat domain comprise an amino acid sequence selected from any one of SEQ ID Nos. 6, 7, 8, 9, 11, 13, 16, 22 and 23. More preferably said designed ankyrin repeat domain comprises an amino acid sequence selected from any one of SEQ ID Nos. 6, 7, 9 and 22.

A preferred virus to be used in the context of the present disclosure is adenovirus of serotype 5. Viruses, other than adenoviruses of serotype 5, can however also be used within the spirit of the present disclosure. For example, such viruses can be engineered to carry the knob of an adenovirus of serotype 5. The recombinant protein disclosed herein, in particular, the recombinant protein comprising DARPin 1D3, may then be used with such viruses.

Therefore, in certain embodiments the present disclosure provides a recombinant protein comprising:
a) a first designed ankyrin repeat domain specifically binding to FAP,
b) a second designed ankyrin repeat domain specifically binding to the knob of an adenovirus, and
c) a trimerization domain,
wherein said designed ankyrin repeat domain binds to the knob of an adenovirus of serotype 5.

It will be understood that also other adenoviral serotypes may be used in the spirit of the present disclosure, including human adenovirus serotype 5 (HAdV-C5), HAdV-C2, HAdV-B3, HAdV-B35, HAdV-D26, as well as hybrids thereof. A list of adenoviruses can be found on the website of the Human Adenovirus Working group (http://hadvwg.gmu.edu). Also, non-human adenoviruses may be used within the scope of the present disclosure, such as the AstraZeneca vaccine chimpanzee adenovirus Y25 (CHAdY25), or non-human adenoviral vectors were developed from bovine (BAd), canine (CAd), chimpanzee (ChAd), ovine (OAd), porcine (PAd), or fowl (FAd).

In certain embodiments, the present disclosure relates to recombinant proteins that comprise a trimerization domain. The trimerization domain is responsible for the formation of trimers. Each monomer of the molecules of the present disclosure comprises a trimerization domain. Principally any trimerization domain may be used, provided it is stable and geometrically fits the knob of the adenovirus used. A preferred trimerization domain is the capsid protein SHP of lambdoid phage 21 (J Mol Biol; 344 (1): 179-93; PNAS 110 (10): E869-77 (2013)).

Therefore, in certain embodiments the present disclosure relates to recombinant proteins comprising
a) a first designed ankyrin repeat domain specifically binding to FAP,
b) a second designed ankyrin repeat domain specifically binding to the knob of an adenovirus, and
c) a trimerization domain,
wherein said trimerization domain is the capsid protein SHP of lambdoid phage 21.

In certain embodiments, the present disclosure relates to recombinant proteins comprising
a) a first designed ankyrin repeat domain specifically binding to FAP,
b) a second designed ankyrin repeat domain specifically binding to the knob of an adenovirus, and
c) a trimerization domain,
wherein said trimerization domain is derived from the capsid protein SHP of lambdoid phage 21.

In certain embodiments, the present disclosure relates to recombinant proteins comprising
a) a first designed ankyrin repeat domain specifically binding to FAP,
b) a second designed ankyrin repeat domain specifically binding to the knob of an adenovirus, and
c) a trimerization domain, wherein said trimerization domain comprises the amino acid sequence of SEQ ID No. 1.

In certain embodiments, the present disclosure relates to recombinant proteins comprising
a) a first designed ankyrin repeat domain specifically binding to FAP,
b) a second designed ankyrin repeat domain specifically binding to the knob of an adenovirus, and
c) a trimerization domain,
wherein said trimerization domain comprises the amino acid sequence of SEQ ID No. 1, and
wherein said first designed ankyrin repeat domain comprises an amino acid sequence selected from any one of SEQ ID Nos. 5-28. Preferably said designed ankyrin repeat domain comprise an amino acid sequence selected from any one of SEQ ID Nos. 6, 7, 8, 9, 11, 13, 16, 22 and 23. More preferably said designed ankyrin repeat domain comprises an amino acid sequence selected from any one of SEQ ID Nos. 6, 7, 9 and 22. Also, other trimerization domains known to the skilled person may be used for the formation or trimers. Without being limited other potential trimerization domains include the trimerization domain involved in collagen folding (Int J Biochem Cell Biol 44:21-32 (2012)), the trimerization domain of T4 phage fibritin (PLOS One 7: e43603 (2012)) or the GCN4-based isoleucine zipper (J Biol Chem 290:7436-42 (2015)).

The trimerization domain is responsible for the formation of the trimeric adapter molecules. The trimers disclosed herein are extraordinarily stable (J Mol Biol (2004) 344:179-93; PNAS (2013) 110 E869-77). In certain embodiments the trimeric adapter molecules of the present disclosure remain intact in SDS gel electrophoresis. In other embodiments the trimeric adapter molecules are not denatured in SDS gel electrophoresis. In other embodiments the trimeric adapter molecules have a half-life in solution of at least one week, preferably at least two week and even more preferably at least one month.

Therefore, in certain embodiments, the present disclosure relates to recombinant proteins comprising
a) a first designed ankyrin repeat domain specifically binding to FAP,
b) a second designed ankyrin repeat domain specifically binding to the knob of an adenovirus, and
c) a trimerization domain,
wherein said trimerization domain has a half-life in solution of at least one week, preferably at least two week and even more preferably at least one month.

The recombinant protein of the present disclosure may also comprise a flexible linker. If the recombinant protein comprising from the N- to the C-terminus
a) a first designed ankyrin repeat domain specifically binding to FAP,
b) a second designed ankyrin repeat domain specifically binding to the knob of an adenovirus, and
c) a trimerization domain,
then said flexible linker is between said first designed ankyrin repeat domain specifically binding to FAP and said second designed ankyrin repeat domain specifically binding to the knob of an adenovirus.

Therefore, in certain embodiments the present disclosure relates to a recombinant protein comprising from the N- to the C-terminus
a) a first designed ankyrin repeat domain specifically binding to FAP,
b) a flexible linker,
c) a second designed ankyrin repeat domain specifically binding to the knob of an adenovirus, and
d) a trimerization domain.

If the recombinant protein comprising from the N- to the C-terminus
a) a first designed ankyrin repeat domain specifically binding to the knob of an adenovirus,
b) a trimerization domain, and
c) a second designed ankyrin repeat domain specifically binding to FAP, then said flexible linker is between said trimerization domain and said second designed ankyrin repeat domain specifically binding to FAP.

Therefore, in certain embodiments the present disclosure relates to a recombinant protein comprising from the N- to the C-terminus
a) a first designed ankyrin repeat domain specifically binding to the knob of an adenovirus,
b) a trimerization domain,
c) a flexible linker, and
d) a second designed ankyrin repeat domain specifically binding to FAP.

Principally any flexible linker can be used within the spirit of the present disclosure. Certain preferred flexible linkers are glycine-serine linkers. A particularly preferred flexible linker is a (Gly₄Ser)₄-linker.

Therefore, in certain embodiments the present disclosure relates to a recombinant protein comprising from the N- to the C-terminus
a) a first designed ankyrin repeat domain specifically binding to FAP,
b) a flexible linker,
c) a second designed ankyrin repeat domain specifically binding to the knob of an adenovirus, and
d) a trimerization domain,
wherein said flexible linker is a glycine-serine linker.

In other embodiments the present disclosure relates to a recombinant protein comprising from the N- to the C-terminus
a) a first designed ankyrin repeat domain specifically binding to the knob of an adenovirus,
b) a flexible linker,
c) a trimerization domain, and
d) a second designed ankyrin repeat domain specifically binding to FAP,
wherein said flexible linker is a (Gly₄Ser)₄-linker.

The recombinant protein of the present disclosure may also comprise a short linker. The short linker is located between the designed ankyrin repeat domain which binds to the knob of an adenovirus and the trimerization domain.

Therefore, in certain embodiments the present disclosure relates to a recombinant protein comprising from the N- to the C-terminus
a) a first designed ankyrin repeat domain specifically binding to FAP,
b) a second designed ankyrin repeat domain specifically binding to the knob of an adenovirus,
c) a short linker, and
d) a trimerization domain.

In other embodiments the present disclosure relates to a recombinant protein comprising from the N- to the C-terminus
a) a first designed ankyrin repeat domain specifically binding to the knob of an adenovirus,
b) a short linker,
c) a trimerization domain, and
d) a second designed ankyrin repeat domain specifically binding to FAP.

The short linker does not necessarily be present. Possible short linkers of the present disclosure are linkers which are no longer than four, no longer than three, no longer than two or only one amino acid long. A preferred short linker is glycine. Another preferred short linker is glycine-alanine. Most preferably the short linker is glycine.

In certain embodiments, the present disclosure relates to a trimeric protein comprising three recombinant proteins as described herein above. In certain embodiments, the present disclosure relates to a trimeric protein consisting of three recombinant proteins as described herein above.

The recombinant proteins of the present disclosure are encoded by nucleic acids. Vectors comprising these nucleic acids are used to transfect cells which express the recombinant proteins.

Therefore, in certain embodiments, the present disclosure relates to a nucleic acid encoding a recombinant protein of the present disclosure. The present disclosure also relates to a nucleic acid encoding a recombinant protein comprising
a) a first designed ankyrin repeat domain specifically binding to FAP,
b) a second designed ankyrin repeat domain specifically binding to the knob of an adenovirus, and
c) a trimerization domain.

In other embodiments, the present disclosure relates to a vector comprising a nucleic acid encoding a recombinant protein of the present disclosure. The present disclosure also relates to a vector comprising a nucleic acid encoding a recombinant protein comprising
a) a first designed ankyrin repeat domain specifically binding to FAP,
b) a second designed ankyrin repeat domain specifically binding to the knob of an adenovirus, and
c) a trimerization domain.

In another embodiments, the present disclosure relates to an adenovirus comprising a nucleic acid encoding a recombinant protein of the present disclosure. In yet another embodiment, the present disclosure relates to an adenovirus comprising a vector comprising a nucleic acid encoding a recombinant protein of the present disclosure. In certain embodiments said adenovirus carries a TAYT mutation. In certain embodiments said adenovirus carries a HVR7 mutation.

In certain embodiments, the present disclosure relates to an adenoviral vector comprising a nucleic acid encoding a recombinant protein comprising
a) a first designed ankyrin repeat domain specifically binding to FAP,
b) a second designed ankyrin repeat domain specifically binding to the knob of an adenovirus, and
c) a trimerization domain.

In certain embodiments, the present disclosure relates to an a recombinant adenovirus displaying a recombinant protein according or a trimeric protein as disclosed herein. In certain embodiments said adenovirus is of adenovirus serotype 5. In other embodiments said adenovirus comprises a knob of an adenovirus of serotype 5.

The recombinant proteins of the present disclosure can be expressed in prokaryotic cells, such as Escherichia coli, and in eukaryotic cells. Preferred eukaryotic cells are CHO cells. Other preferred eukaryotic cells are HEK293 cells, HEK293-T cells, HEK293-F cells, CHO-S cells and Sf9 cells. Therefore, in certain embodiments the present disclosure provides a eukaryotic cell expressing the recombinant protein of the present disclosure. In certain other the present disclosure provides a CHO cell expressing the recombinant protein of the present disclosure. In certain embodiments, the present disclosure relates to a eukaryotic cell expressing a recombinant protein comprising
a) a first designed ankyrin repeat domain specifically binding to FAP,
b) a second designed ankyrin repeat domain specifically binding to the knob of an adenovirus, and
c) a trimerization domain.

In certain embodiments, the present disclosure relates to a CHO cell expressing a recombinant protein comprising
a) a first designed ankyrin repeat domain specifically binding to FAP,
b) a second designed ankyrin repeat domain specifically binding to the knob of an adenovirus, and
c) a trimerization domain.

The recombinant proteins of the present disclosure, the nucleic acids encoding the recombinant proteins and trimeric proteins of the present disclosure, the vectors containing the nucleic acids of the present disclosure and the recombinant adenoviruses containing the recombinant proteins, the nucleic acids or the vectors of the present disclosure have numerous uses, such as the use in an adenoviral delivery system. Therefore, in certain embodiments the present disclosure provides the use of the recombinant proteins of the present disclosure in an adenoviral delivery system. In other embodiments the present disclosure provides the use of the nucleic acids encoding the recombinant proteins of present disclosure in an adenoviral delivery system. In other embodiments the present disclosure provides the use of the vectors containing the nucleic acids of the present disclosure in an adenoviral delivery system. In other embodiments the present disclosure provides the use of the adenoviruses containing the recombinant proteins, the nucleic acids or the vectors of the present disclosure in an adenoviral delivery system.

The recombinant proteins of the present disclosure, the nucleic acids encoding the recombinant proteins and trimeric proteins of the present disclosure, the vectors containing the nucleic acids of the present disclosure and the recombinant adenoviruses containing the recombinant proteins, the nucleic acids or the vectors of the present disclosure can also be used for the transduction of fibroblasts. Therefore, in certain embodiments the present disclosure provides the use of the recombinant proteins of the present disclosure for the transduction of fibroblasts.

The recombinant proteins of the present disclosure, the nucleic acids encoding the recombinant proteins and trimeric proteins of the present disclosure, the vectors containing the nucleic acids of the present disclosure and the recombinant adenoviruses containing the recombinant proteins, the nucleic acids or the vectors of the present disclosure can also be used in medicine. Therefore, in certain embodiments the present disclosure provides the use of the recombinant proteins of the present disclosure in medicine. In other embodiments the present disclosure provides the use of the nucleic acids encoding the recombinant proteins of the present disclosure in medicine. In other embodiments the present disclosure provides the use of the vectors containing the nucleic acids of the present disclosure in medicine. In other embodiments the present disclosure provides the use of the adenoviruses containing the recombinant proteins, the nucleic acids or the vectors of the present disclosure in medicine.

In certain embodiments the present disclosure provides a method to treat a patient, said method comprising administering to a patient in need of a recombinant protein of the present disclosure. In certain embodiments the present disclosure provides a method to treat a patient, said method comprising administering to a patient in need of a nucleic acid encoding a recombinant protein of the present disclosure. In certain embodiments the present disclosure provides a method to treat a patient, said method comprising administering to a patient in need thereof a vector containing a nucleic acid of the present disclosure. In certain embodiments the present disclosure provides a method to treat a patient, said method comprising administering to a patient in need thereof a recombinant adenovirus containing a recombinant protein, a nucleic acid or a vector of the present disclosure.

Principally, the recombinant proteins of the present disclosure, the nucleic acids encoding the recombinant proteins of the present disclosure, the vectors containing the nucleic acids of the present disclosure, the recombinant adenovirus displaying or containing the recombinant proteins, the nucleic acids or the vectors of the present disclosure, and the eukaryotic cells containing the recombinant adenovirus or set of recombinant adenoviruses of the present disclosure can be used in the treatment or prevention of any disease or disorder. Particularly preferred is the treatment of cancer.

### EXAMPLES

### Example 1: General Experimental Procedures

### Cell Lines and Mice

The human embryonic skin fibroblast cell line Detroit 551 (D551; ATCC, Order no. CCL-110) was maintained in complete DMEM media (DMEM-high glucose supplemented with 10% (v/v) fetal bovine serum (FBS), 100 U/ml penicillin and 100 μg/mL streptomycin). The Detroit 551 cell line endogenously expresses human FAP.

The mouse embryonic fibroblast cell line NIH3T3 (available from ATCC, Order No. CRL-1658) and NIH3T3mFAP were generated by lentiviral transduction as described previously (Cancer Immunol Res (2014) 2 (2): 154-166). The NIH3T3 cell line transduced to express murine FAP is referred to as NIH3T3mFAP. Cells were maintained in complete RPMI media (RPMI1640 GlutaMax supplemented with 10% (v/v) FBS, 100 U/ml penicillin and 100 μg/mL streptomycin).

The human fibrosarcoma cell line HT1080 (available from ATCC, Order no. CCL-121) and HT1080hFAP were generated through stable transfection as described previously (J Transl Med (2013) 11:187. Cell lines were maintained in complete RPMI media containing 200 μg/mL G418 and 150 μg/mL Hygromycin B. The HT1080 cell line stably transfected with human FAP is referred to as HT1080hFAP.

The human gastric carcinoma cell line (ATCC, Order no. CRL 5822) was maintained in complete RPMI media.

Fox Chase SCID/beige mice (CB17.Cg-PrkdcscidLystbg-J/Crl) used for in vivo studies were obtained from Charles River. All animal experiments were performed in accordance with the Swiss animal protection law and with approval of the Cantonal Veterinary Office (Zurich, Switzerland).

### Viral Vector Generation

The replication-deficient HAdV-C5 contains an E1/E3 deletion and 4 mutations in the HVR7 (1421G, T423N, E424S and L426Y) and was generated as previously described (Nat. Commun. 9, 450 (2018)) or ordered from Vector Biolabs (Malvern, PA/USA).

### Expression and Purification of the Recombinant Proteins and Adapter Molecules

DARPins were cloned into the pQlq backbone containing an N terminal His₆ tag and a C terminal FLAG tag. Adapters were cloned into the pQlq backbone containing an N terminal His₁₀ tag with a 3C protease cleavage site. For DNA propagation, the E. coli XL1 Blue strain was transformed with the corresponding plasmid.

DARPins and adapters were either expressed in small-scale format (1 mL culture volume) in the E. coli XL1 Blue strain, or in large-scale format (500 mL culture volume) in the E. coli BL21 strain, as described by Dreier et. al (J Mol Biol. (2011); 405 (2): 410-426). For small-scale format, protein purification via immobilized metal ion affinity chromatography (IMAC) was performed using a HisPur™ Cobalt Spin Plates (Thermo Fisher Scientific) equilibrated with equilibration buffer (50 mM Na₂HPO₄/NaH₂PO₄, 300 mM NaCl, pH 7.4). Bound proteins were washed with wash buffer (50 mM Na₂HPO₄/NaH₂PO₄, 300 mM NaCl, 15 mM imidazole, pH 7.4), eluted in elution buffer (50 mM Na₂HPO₄/NaH₂PO₄, 300 mM NaCl, 250 mM imidazole, pH 7.4), and further buffer-exchanged to PBS (pH 7.4) using a Pall AcroPrep™ filter plate (Pall). For large-scale format, proteins were purified via IMAC as described by Dreier et. al (J Mol Biol. (2011); 405 (2): 410-426).

For in vivo experiments, purified adapters were incubated with 3C protease for removal of the His 10 tag during dialysis against PBS (pH 7.4) for buffer exchange. Adapters were then further purified via size exclusion chromatography (SEC) using a Superdex™ 200 10/300 GL column (GE Healthcare). Prior to in vivo injection into mice, purified adapters were confirmed to comply with the recommendation by the Food and Drug Administration (FDA) on the maximal endotoxin content for injectable products.

Purified protein was shock-frozen in liquid nitrogen and stored at −80° C. until usage.

ELISA with Murine FAP

Recombinant mFAP (R&D Systems), and recombinant maltose-binding protein (MBP; inhouse production) for a positive control, were used for a DARPin binding analysis via ELISA. Target protein was coated overnight (O/N) at 4° C. on MaxiSorp™ 96-well plates (Nunc). Blocking was performed for 2 h at RT using ELISA-blocking buffer (PBS containing 0.5% (w/v) BSA). Plates were washed with ELISA-PBS-T (PBS containing 0.05% (v/v) Tween 20). Samples were diluted (100 nM DARPin concentration) in ELISA-PBS-TB (ELISA PBS T containing 0.5% (w/v) BSA) and incubated for 1 h at 4° C. while shaking. DARPin detection was performed with rabbit anti-FLAG antibody (GenScript, A01868; 1:5,000) and goat anti-rabbit-AP antibody (Sigma Aldrich, A3687; 1:10,000), both diluted in ELISA-PBS-TB and incubated for 1 h at 4° C. while shaking. Substrate solution consisting of 3 mM p-nitrophenyl phosphate (pNPP) diluted in pNPP buffer (50 mM NaHCO₃, 50 mM MgCl₂) was used for final detection. A Tecan Infinite M1000 plate reader (Tecan) was used to measure the absorbance at 405 nm.

In vitro transduction assays 1×10⁴ or 5×10⁴ cells were seeded per well of a cell culture 96-well or 24-well plate, respectively. Adenoviral vector was pre-incubated for 1 h at 4° C. with the corresponding FAP adapter (anti-human FAP or anti-mouse FAP, termed “retargeting adapters”), or a blocking adapter containing DARPin E3_5 (J Mol Biol (2003) 332:489-503) (termed “non-binding adapter”). A tenfold molar excess of adapter over knob was used during pre-incubation. As a control, adenoviral vector without adapter (termed “untargeted vector”) was incubated with PBS. For transduction, cell media was changed to fresh complete media and (retargeted) AdV vector was added to the cells at a multiplicity of infection (MOI; referring to optical viral particles) of 100-1000. Cells were incubated for 4 h (unless stated otherwise) at 37° C. and 5% CO₂ before performing another media change to remove the viral vector. Cells were further incubated for 20-26 h at 37° C. and 5% CO₂, before being harvested and analyzed via flow cytometry for fluorescent reporter gene expression.

### Animal Experiments

Fox Chase SCID/beige mice (CB17.Cg-PrkdcscidLystbg-1/Crl) were obtained from Charles River and housed under specific-pathogen-free conditions at the Laboratory Animal Services Center, University of Zurich. Eight-to-nine weeks old female mice were used for in vivo retargeting experiments.

For tumor engraftment, 1×10⁶ NCI-N87 and 1×10⁴ NIH3T3mFAP cells in 100 μL PBS containing 50% Matrigel (Corning) were subcutaneously injected into the left flank of the mouse. Tumors were measured with a caliper and tumor volumes calculated via V=0.5×length×width×width. After tumor establishment, mice were intratumorally injected with 50 μL retargeted or untargeted vector (3×10⁹ PFU in PBS) encoding TdTomato. For FAP-retargeted AdV, vector had been pre-incubated with the corresponding adapter (tenfold molar excess of adapter over knob) diluted in PBS for 1 h on ice. Tumors were harvested 72 h post injection for analysis via flow cytometry.

### Flow Cytometry

For in vitro transduction assays, cells were harvested by trypsinization, centrifuged at 500 g for 5 min, and resuspended in FACS-buffer (PBS containing 1% (w/v) bovine serum albumin (BSA) and 0.1% (w/v) azide). If indicated, cells were stained with appropriate antibodies (diluted in FACS-buffer) for 20 min on ice in the dark. After washing twice with FACS-buffer, cells were resuspended in fixation buffer (PBS containing 2% (w/v) paraformaldehyde (PFA)) and fixed for 10 min at room temperature. After a final wash step, cells were resuspended in FACS-buffer and stored at 4° C. until being analyzed at the flow cytometer. For transduction analysis of harvested tumors from mice, tumors were minced with a scalpel, incubated in digestion media (RPMI1640 GlutaMax supplemented with 2% (v/v) FBS, 1 mg/ml collagenase IV (Gibco), 0.5 mg/ml hyaluronidase (ITW Reagents), and 0.5 mg/ml DNase I (Merck)) for 30 min at 37° C., and passed through a 70 μm mesh to yield a single-cell suspension. Cells were collected by centrifugation (500 g for 5 min at 4° C.) and washed with ice-cold PBS. Subsequently, red blood cell (RBC) lysis was performed using ACK Lysing Buffer (Thermo Fisher Scientific) following the manufacturer's instructions. ACK Lysing Buffer was diluted by adding a tenfold excess of PBS containing 5 mM EDTA, followed by an incubation for 10 min on ice. Cells were then washed with ice-cold PBS and subjected to a live/dead staining using the LIVE/DEAD™ Fixable Violet Dead Cell Stain Kit (Invitrogen). Then, IgG Fc receptors were blocked using TruStainFcX™ PLUS (BioLegend) followed by the antibody staining and subsequent fixation procedure described above.

For multicolor flow cytometry, single-stained controls or UltraComp eBeads Plus (Thermo Fisher Scientific) were used for compensation. Fluorescence minus one (FMO) and further proper controls were included in each experiment.

Flow cytometers used herein included BD FACSCanto™ II, BD LSRFortessa™, BD FACSymphony™ 5L (all BD Biosciences), and Cytek Aurora 5L (Cytek Biosciences). Flow cytometry analysis was performed using FlowJo™ software (BD Biosciences).

### Example 2: Isolation of DARPins Targeting Human FAP

Using ribosome display (Proc Natl Acad Sci USA (1997) 94:4937-42), ankyrin repeat proteins with a desired specificity can be selected from DARPin libraries similar as described by Binz et al. (Nat Biotechnol (2004) 22:575-82). The present disclosure utilized such an approach to isolate ankyrin repeat proteins with specificity for human FAP.

Initially 380 binders were isolated which were then screened in a flow cytometry-assisted cell-based binding assay with FAP-positive HT1080hFAP and FAP-negative HT1080 cells. Out of the initial 380 binders, more than two dozen specific binders were selected from this screen. Sequencing revealed that some of the binders had identical amino acid sequences. Amino acid sequences of unique binders are shown in SEQ ID Nos. 5-28. These binders were subjected to additional assays and tests, including SDS-PAGE, size exclusion chromatography, and retesting in cell-based binding assays with FAP-positive and FAP-negative cells. Thereafter, 10 DARPins with a high specificity for human FAP were selected. These 10 DARPins were used to generate adapter molecules as described herein above.

### Example 3: Cross-Reactivity to Mouse FAP

The anti-human FAP DARPins were tested for cross-reactivity to mouse FAP in cell-based binding assays, and via ELISA utilizing recombinant mouse FAP.

Of the anti-human FAP DARPins tested, one was cross-reactive to mouse FAP in both assays, namely DARPin #6 (SEQ ID No. 9). This cross-reactive DARPin was used later for in vivo xenograft experiments in mice.

### Example 4: Vectors Displaying Anti-Human FAP Adapters Transduce Human Cells Stably Transfected to Express Human FAP

HT1080 and HT1080hFAP cells were transduced with human FAP-targeted adenoviral vectors. As controls, a vector displaying a non-binding adapter and an untargeted vector were used.

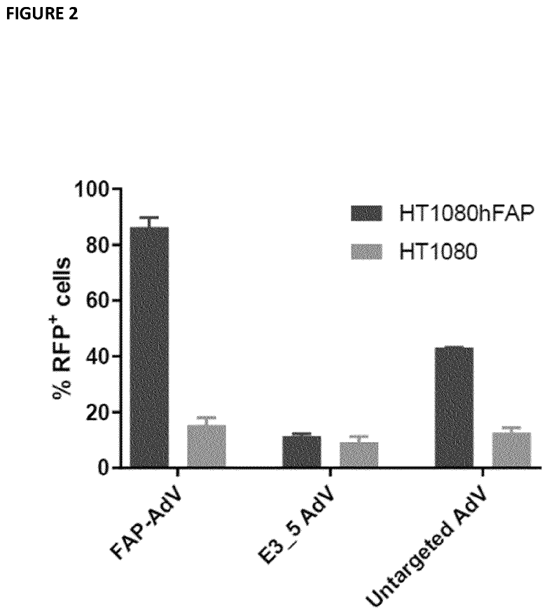

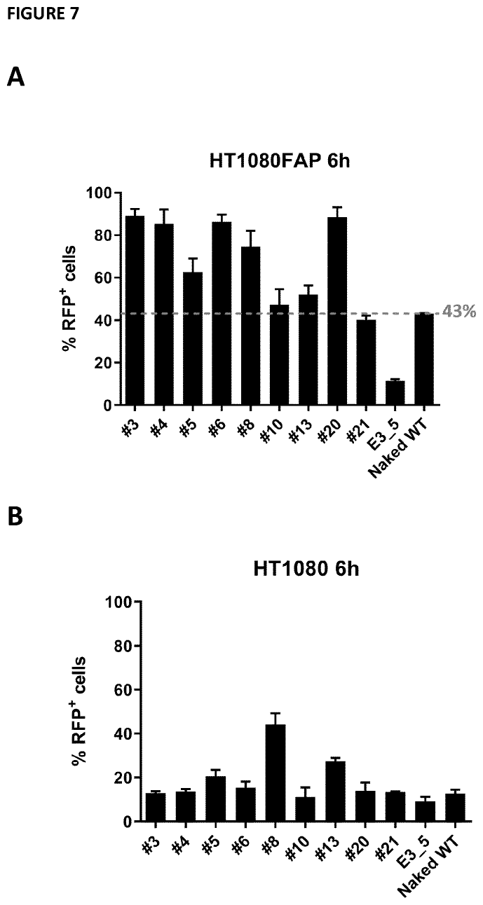

Transduction was quantified via cellular expression of the fluorescent reporter protein iRFP (encoded by the adenoviral vector), and was detected via flow cytometry. Results are shown in FIG. 2 and FIG. 7. Targeted vectors showed a significant increase of transduction in HT1080 cells expressing human FAP. The untargeted vector also showed some level of transduction in HT1080hFAP cells, although significantly less pronounced than the targeted vectors. The vector displaying the non-binding adapter did not show any relevant levels of transduction. Highest transduction levels were observed with binders comprising SEQ ID Nos. 6, 7, 8, 9, 11 and 22, and moderate transduction levels with binders comprising SEQ ID Nos. 13, 16, and 23. Of note, FAP-specific binding of the DARPin used to construct the retargeting adapter was not necessarily associated with an efficient and specific transduction of target cells by the adenoviral vector. Whilst all forementioned adapters specifically bound to FAP, only adapters #3 (SEQ ID No. 6), #4 (SEQ ID No. 7), #6 (SEQ ID No. 9) and #20 (SEQ ID No. 22) showed to mediate a highly-efficient and specific adenoviral transduction of target cells (see FIGS. 7A and B). In summary, it was demonstrated that certain adapters targeting anti-human FAP significantly increase the transduction efficiency of adenoviral vectors to cells expressing human FAP.

### Example 5: Vectors Displaying Anti-Human FAP Adapters Transduce Human Fibroblasts which Endogenously Express Human FAP

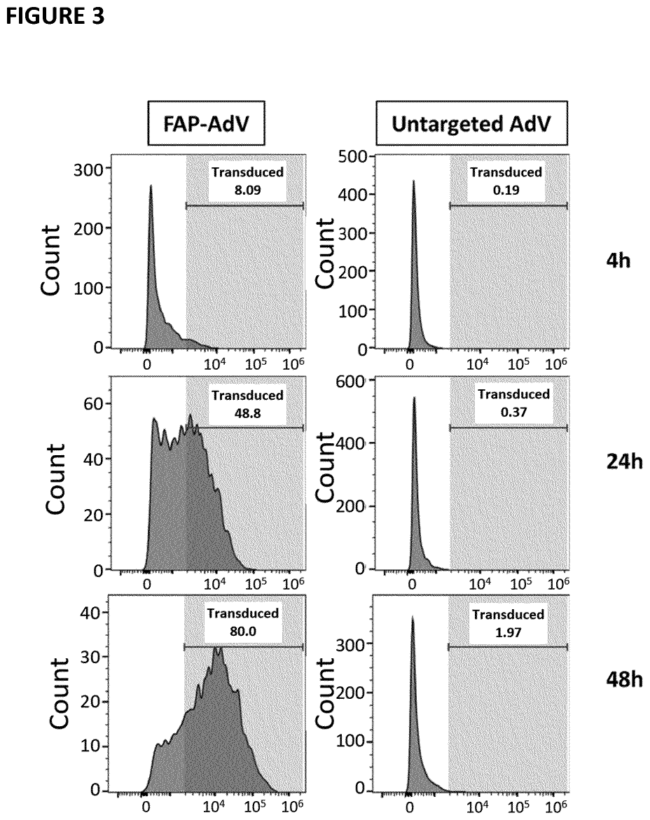

In this experiment the Detroit 551 cell line, which endogenously expresses human FAP, was transduced with adenoviral vectors targeted against human FAP and with untargeted adenoviral vectors. Transduction was measured over time by flow cytometry detecting the cellular expression of a fluorescent reporter protein encoded by the vector. Results are shown in FIG. 3.

Already after 4 hours the targeted vector showed a transduction rate of almost 10%, as compared to below 1% for the untargeted vector. After 24 hours the transduction rate of the targeted vector reached 48%, and exceeded 80% after 48 hours, whereas the transduction rate of the untargeted vector remained at around 1%.

In summary, it was demonstrated that human fibroblasts can be efficiently transduced with adenoviral vectors targeting human FAP.

### Example 6: Vectors Displaying Anti-Murine FAP Adapters Transduce Mouse Fibroblasts Engineered to Express Mouse FAP

NIH3T3 and NIH3T3mFAP cells were transduced with targeted adenoviral vectors against mouse FAP. As controls, vectors with non-binding adapters and untargeted vectors were used.

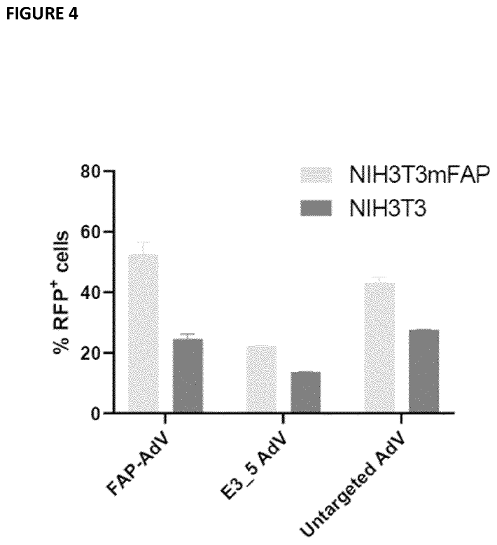

Transduction was quantified via iRFP expression. Results are shown in FIG. 4. Targeted vectors showed a clear increase of transduction in NIH3T3 cells expressing murine FAP. Untargeted vectors also showed some level of transduction in NIH3T3mFAP cells, although less pronounced than the targeted vector. Vectors with non-binding adapters did not show any relevant levels of transduction. In summary, it was demonstrated that adapters targeting mouse FAP increase the transduction efficiency of adenoviral vectors to mouse fibroblasts.

### Example 7: Anti-Murine FAP-Based Adapters Mediate In Vivo Transduction of Fibroblasts in a Xenograft Mouse Model

Fox Chase SCID/beige mice were subcutaneously injected into the flank with NCI-N87 and NIH3T3mFAP cells. After tumor establishment, mice were intratumorally injected with targeted (FAP-AdV) or untargeted (untargeted AdV) vector encoding TdTomato. Three days post injection, mice were sacrificed, and tumors were harvested for subsequent analysis via flow cytometry.

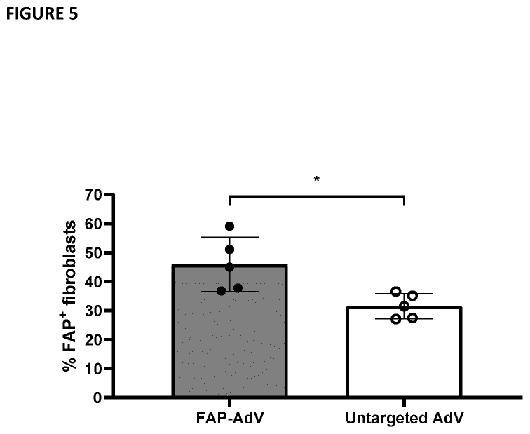

Results are shown in FIG. 5. Analysis of transduced FAP⁺ fibroblasts in the TME revealed a significant increase in target cell transduction with FAP-retargeted vector (FAP-AdV) compared to untargeted vector (untargeted AdV).

In summary, it was demonstrated that adapters targeting mouse FAP increase the transduction efficiency of adenoviral vectors to mouse fibroblasts in vivo.

### Example 8: In Vivo Delivery of Anti-Cancer Therapeutics Via Anti-FAP Adapters

In this experiment it was tested if anti-FAP adapters can be used to efficiently deliver anti-cancer therapeutics into cells. HER2-overexpressing NCI-N87 tumor cells and NIH3T3mFAP cells were co-injected subcutaneously into the flank of SCID/beige mice for tumor establishment. At a tumor volume of 50 mm³, mice were treated intratumorally with FAP-retargeted Ad5 (DARPin #6, SEQ ID No. 9) encoding Trastuzumab (FAP-Ad5-TZB; n=5), or one single dose of 200 μg Herceptin (n=3), or three doses of 200 μg Herceptin (n=3), or PBS (n=5).

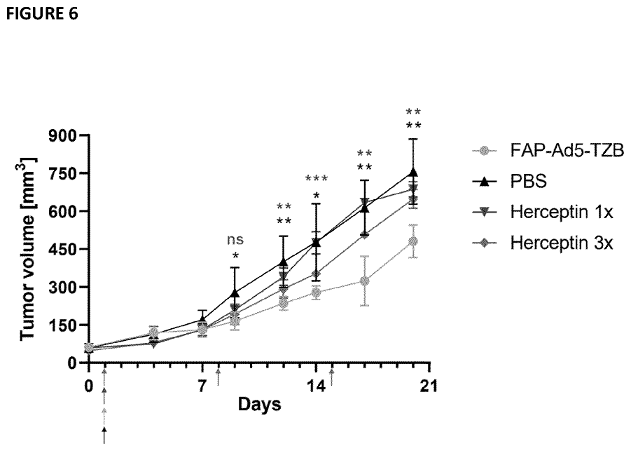

Results are shown in FIG. 6. Mice treated with anti-FAP-retargeted Ad5 encoding Trastuzumab clearly showed less tumor volume. Expression of Trastuzumab was also confirmed via immunofluorescence staining.

1. A recombinant protein capable of mediating the transduction of FAP-positive cells comprising
a) a first designed ankyrin repeat domain specifically binding to FAP,
b) a second designed ankyrin repeat domain specifically binding to the knob of an adenovirus, and
c) a trimerization domain.

2. The recombinant protein according to claim 1, wherein said FAP-positive cells are cancer cells, non-cancerous cells of the tumor microenvironment or fibroblasts.

3. The recombinant protein according to claim 1, wherein said first designed ankyrin repeat domain comprises the amino acid sequence of any one of SEQ ID Nos. 6, 7, 9 or 22.

4. The recombinant protein according to claim 1, wherein said first designed ankyrin repeat domain is cross-reactive with mouse FAP.

5. The recombinant protein according to claim 1, wherein said recombinant protein comprises from the N- to the C-terminus said first designed ankyrin repeat domain specifically binding to FAP, said second designed ankyrin repeat domain specifically binding to the knob of an adenovirus and said trimerization domain.

6. The recombinant protein according to claim 1, wherein said designed ankyrin repeat domain that specifically binds to the knob of an adenovirus comprises the amino acid sequence of SEQ ID No. 2.

7. The recombinant protein according to claim 1, wherein said trimerization domain is or is derived from the capsid protein SHP of lambdoid phage 21.

8. The recombinant protein according to claim 6, wherein said trimerization domain comprises the amino acid sequence of SEQ ID No. 1.

9. A nucleic acid encoding a recombinant protein according to claim 1.

10. A trimeric protein comprising three recombinant proteins according to claim 1.

11. A recombinant adenovirus displaying a recombinant protein according to claim 1.

12. The recombinant adenovirus according to claim 11, wherein said adenovirus is of adenovirus serotype 5 or wherein said adenovirus comprises a knob of an adenovirus of serotype 5.

13-14. (canceled)

15. A recombinant protein comprising an ankyrin repeat domain specifically binding to FAP, wherein said ankyrin repeat domain comprises an amino acid sequence selected from the group consisting of SEQ ID No. 5-28.

16. A recombinant adenovirus displaying a recombinant protein according to claim 10.

17. The recombinant adenovirus according to claim 16, wherein said adenovirus is of adenovirus serotype 5 or wherein said adenovirus comprises a knob of an adenovirus of serotype 5.

18. A method for transducing FAP-positive cells, said method comprising administering to the cells a recombinant protein of claim 1 or a trimeric protein or a recombinant adenovirus comprising said recombinant protein.

19. The method of claim 18 wherein said FAP-positive cells are cancer cells, non-cancerous cells of the tumor microenvironment or fibroblasts.
